(19) United States
(12) Patent Application Publication
(10) Pub. No.: US 2025/0275802 A1
(43) Pub. Date: Sep. 4, 2025

(54) DEVICES AND RELATED METHODS FOR ELECTROSURGERY

(71) Applicant: Dignity Health, San Francisco, CA (US)

(72) Inventor: Benjamin Hendricks, San Francisco, CA (US)

(21) Appl. No.: 18/857,992

(22) PCT Filed: May 1, 2023
(86) PCT No.: PCT/US2023/020614

§ 371 Date: Oct. 18, 2024

Related U.S. Application Data
(60) Provisional application No. 63/336,577, filed on Apr. 29, 2022.

(51) Int. Cl. A61B 18/12; A61B 17/00; A61B 18/00; A61B 18/16

(52) U.S. Cl. CPC A61B 18/1206; A61B 18/16; A61B 2017/00199; A61B 2018/00595; A61B 2018/00601; A61B 2018/00642; A61B 2018/00702; A61B 2018/00732; A61B 2018/0075; A61B 2018/00755; A61B 2018/126

(57) ABSTRACT

A system provides an electrosurgical tool that delivers an electrosurgical waveform that can be optimized for electrosurgical tasks such as cutting and cauterization of various tissues. The electrosurgical waveform can include a first electrosurgical waveform and a second electrosurgical waveform for simultaneous delivery at respective stimulating electrodes of a stimulating pathway. In some examples, the second electrosurgical waveform is phase-shifted by 180-degrees to minimize deep penetration of current through tissue. The electrosurgical tool provides a relief pathway for further minimization of deep penetration of current through tissue, and can also enable adaptive adjustment of an impedance of the relief pathway, the stimulating pathway, and/or adjustment of one or more parameters of the electrosurgical waveform.

### CROSS-REFERENCE TO RELATED APPLICATIONS

This is a PCT Patent Application that claims benefit to U.S. Provisional Patent Application Ser. No. 63/336,577 filed 29 Apr. 2022, which is herein incorporated by reference in its entirety.

### FIELD

The present disclosure generally relates to surgical devices, and in particular, to devices and associated methods for electrosurgery applications.

### BACKGROUND

Electrosurgery may be described as the controlled delivery of high-frequency waveforms, or currents, for the purpose of altering local tissues. Bipolar coagulation was introduced in 1940 and the first major commercial system adopted aperiodic waveforms (as in spark-gap generators). The goal with use of the bipolar approach is to restrain the electrical energy dispersion within the tissue to which energy is being applied. This aberrant spread of thermal energy poses the risk for tissue injury, particularly to sensitive neural tissue when dealing with cerebral applications.

It is with these observations in mind, among others, that various aspects of the present disclosure were conceived and developed.

### BRIEF DESCRIPTION OF THE DRAWINGS

FIG. 1 is a simplified diagram showing a system for electrosurgery including an electrosurgical tool, a waveform generator, and a processor;

FIGS. 2A and 2B are a pair of illustrations showing an example of the electrosurgical tool of FIG. 1, with FIG. 2A showing an overview of the electrosurgical tool and with FIG. 2B showing an enlarged view of an example tissue interface of the electrosurgical tool of FIG. 2A;

FIG. 3A is a graphical representation showing an example electrosurgical waveform for application to tissue by the system of FIG. 1 having a first electrosurgical waveform and a second electrosurgical waveform, the second electrosurgical waveform begin phase-shifted by 180 degrees relative to the first electrosurgical waveform;

FIGS. 3B and 3C are a pair of simplified schematic diagrams showing application of the electrosurgical waveform to tissue, where FIG. 3B shows current flowing in a first direction corresponding with intervals denoted by “3B” in FIG. 3A and where FIG. 3C shows current flowing in a second direction corresponding with intervals denoted by “3C” in FIG. 3A;

FIGS. 4A-4C are a series of graphical representations showing results for validation of the stimulating waveform shown in FIGS. 3A-3C, indicating that simultaneous application of the first electrosurgical waveform and the phase-shifted second electrosurgical waveform results in a greater magnitude of energy being delivered to target tissue while reducing spread of energy to surrounding tissues;

FIG. 5A is a graphical representation showing a first example electrosurgical waveform constructed by summation of two individual “sine” waveforms having different parameters;

FIG. 5B is a graphical representation showing an individual waveform used for construction of the first example electrosurgical waveform;

FIG. 5C is a graphical representation showing a second example electrosurgical waveform constructed by summation of more than two individual “sine” waveforms having different parameters;

FIGS. 6A-6C are a series of graphical representations respectively showing third, fourth, and fifth example electrosurgical waveforms constructed from two individual waveforms and combined using amplitude modulation (AM) methods;

FIG. 7A is a graphical representation showing a sixth example electrosurgical waveform constructed from summation of an individual sine waveform and an individual square waveform;

FIG. 7B is a graphical representation showing a sixth example electrosurgical waveform constructed from summation of an individual sine waveform and an individual sawtooth waveform;

FIG. 8 is a graphical representation showing peak-to-peak voltage measurement in tissue with respect to changes in frequency of the electrosurgical waveform as measured through an experimental setup for evaluating how frequency affects spread of energy through tissue;

FIG. 9 is a simplified diagram showing incorporation of feedback by the system of FIG. 1;

FIGS. 10A-10C are a series of simplified schematic diagrams showing a stimulating path and a relief path of the electrosurgical tool of FIG. 2A in a first “stimulating” configuration (FIG. 10A), a second “partial diversion” configuration (FIG. 10B) and a third “full diversion” configuration (FIG. 10C);

FIGS. 10D-10F are a series of simplified schematic diagrams showing alternative arrangements of the stimulating path and a relief path of the electrosurgical tool of FIG. 2A, where FIG. 10D shows the stimulating path belonging to a single tine, where FIG. 10E shows the stimulating pathway belonging to a single contact of a single tine and a remaining contact along the single tine belonging to the relief pathway, and FIG. 10F shows another example of the stimulating path of FIG. 10E with contacts along the opposing tine being turned “off” (e.g., through application of a high impedance);

FIGS. 11A-11C are a series of illustrations showing the tissue interface of FIG. 2B respectively corresponding with the simplified schematic diagrams of FIGS. 10A-10C;

FIGS. 11D-11F are a pair of illustrations showing alternative assignments of contacts of the tissue interface of FIG. 2B, respectively corresponding with the simplified schematic diagrams of FIGS. 10D-10F;

FIGS. 12A and 12B are a pair of simplified schematic diagrams showing fixed impedance components and variable impedance components of the system of FIG. 1 that enable the functionalities shown in FIGS. 10A-10E;

FIG. 13 is a simplified schematic diagram showing a grounding configuration module of the system of FIG. 1;

FIG. 14A is an illustration showing the tissue interface of FIG. 2B having an irrigation line;

FIG. 14B is a simplified block diagram corresponding with the tissue interface of FIG. 14A that shows the irrigation line being in communication with a processor for feedback-initiated irrigation and/or control-input irrigation;

FIGS. 15A and 15B are a pair of illustrations showing an alternative tissue interface configuration of the electrosurgical tool of FIG. 2A where relief contacts are positioned along an external surface of the tissue interface and where stimulating contacts are positioned along an internal surface of the tissue interface;

FIGS. 16A-16D are a series of illustrations showing an alternative tissue interface configuration of the electrosurgical tool of FIG. 2A having a radial arrangement, where FIGS. 16B-16D correspond with respective scenarios shown in FIGS. 11A-11C;

FIG. 17A-17D are a series of illustrations showing progression of a time-varying tissue interface configuration of the electrosurgical tool of FIG. 2A;

FIGS. 18A and 18B are a pair of a process flow charts showing an example method for electrosurgery by the systems outlined herein; and

FIG. 19 is a simplified diagram showing an example computing device for implementation of the system of FIG. 1.

Corresponding reference characters indicate corresponding elements among the view of the drawings. The headings used in the figures do not limit the scope of the claims.

### DETAILED DESCRIPTION

A system and associated methods for precision electrosurgery are described herein. The system provides an electrosurgical tool that delivers an electrosurgical waveform that can be optimized for cutting and cauterization of various tissues. In one aspect, the electrosurgical tool provides a multi-polar contact configuration with a relief pathway for minimization of deep penetration of current through tissue, and can also provide various sensors that enable adaptive adjustment of an impedance of the relief pathway and/or one or more parameters of the electrosurgical waveform.

The system can further include a waveform generator in communication with the electrosurgical tool and a processor that generates the electrosurgical waveform for delivery to tissue. In one aspect, the waveform generator and processor are operable for optimization of the electrosurgical waveform, particularly for optimization of a crest factor, amplitude, and/or frequency of the electrosurgical waveform for precise cutting and cauterization of tissues. The system can, for example, generate an electrosurgical waveform by summation of one or more waveforms of variable shape, amplitude, and frequency. The processor can determine parameters for one or more individual waveforms that can be combined into an electrosurgical waveform exhibiting electrosurgical properties that are optimal or otherwise conducive for the specific electrosurgical task (e.g., a target crest factor, shape, peak-to-peak magnitude, amplitude, and/or frequency). The waveform generator can generate the electrosurgical waveform according to the parameters determined by the processor for delivery to tissue at the electrosurgical tool. In a further aspect, the electrosurgical waveform applied to tissue by the system can include a 180-degree phase shift for reducing unintentional spread of energy to surrounding tissues (e.g., the electrosurgical waveform can be applied at a first tine of the electrosurgical tool, and a 180-degree phase-shifted version of the electrosurgical waveform can be simultaneously applied at a second tine of the electrosurgical tool). In other examples, phase shifting by other values/amounts may be applied (e.g., phase-shifted by 45 degrees, 90 degrees, another suitable value, etc.).

The system can also monitor operation of the electrosurgical tool, and can apply mitigation strategies to improve efficacy of the electrosurgical tool and reduce damage to surrounding tissues based on the measurements. Mitigation strategies can include adjusting the parameters for construction of the electrosurgical waveform, varying an impedance of the relief pathway and/or a stimulating pathway of the electrosurgical tool, and alternating which contacts of the electrosurgical tool deliver the electrosurgical waveform. In one example, coagulum buildup can introduce excessive impedance at the stimulating pathway of the electrosurgical tool that can exceed an impedance of the relief pathway—in such a case, current may be diverted through the relief pathway to prevent damage to surrounding tissues, enhance efficiency, and provide an opportunity for a practitioner to remove the coagulum buildup.

### System Overview

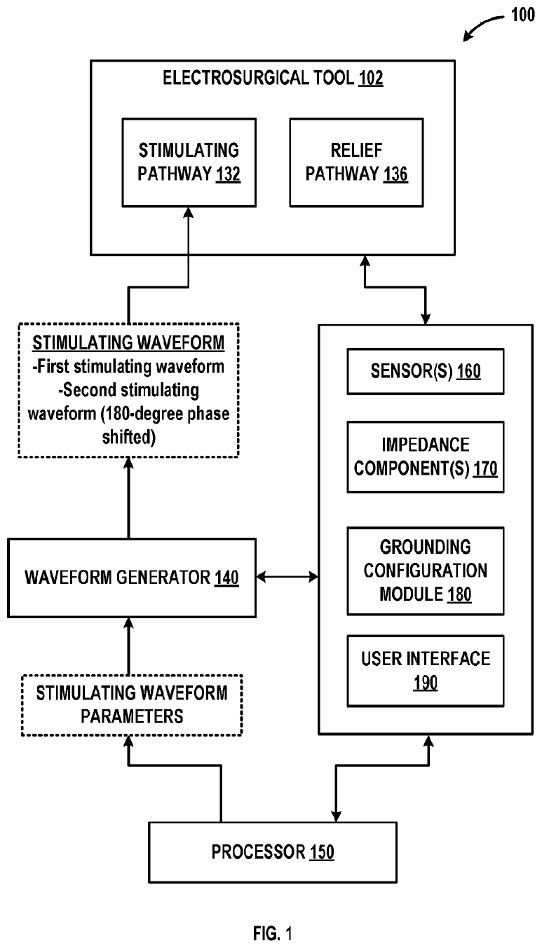

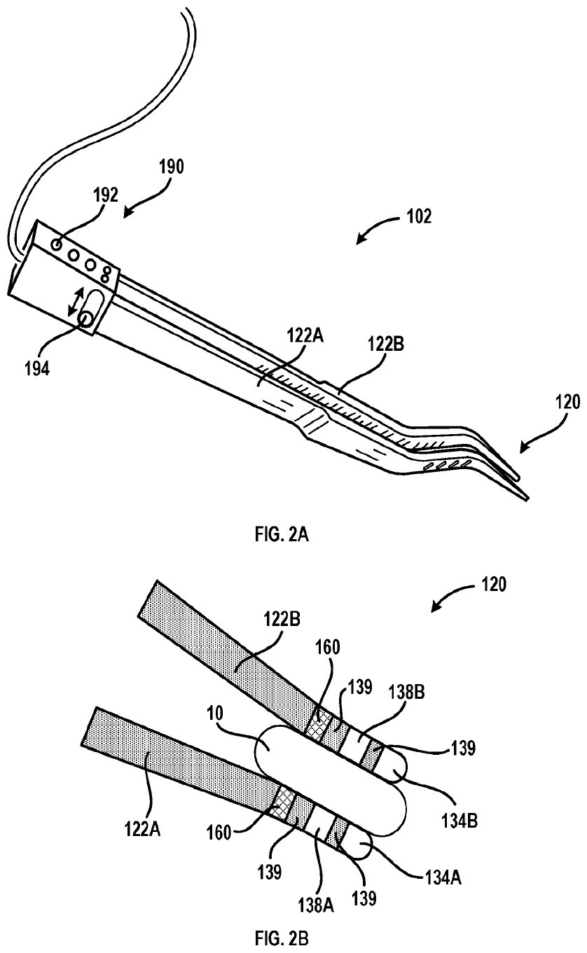

Referring to FIG. 1, a system 100 for precision electrosurgery includes an electrosurgical tool 102 in communication with a waveform generator 140 for application of an electrosurgical waveform to tissues for the purposes of cutting and coagulation. In some embodiments, the system 100 can further include a processor 150 (e.g., of a computing device) that communicates with the electrosurgical tool 102 and/or the waveform generator 140 to perform functionalities such as determining parameters for generation of the electrosurgical waveform and monitoring operation of the electrosurgical tool 102. In some examples, with additional reference to FIGS. 2A and 2B, the electrosurgical tool 102 can be a bipolar electrosurgical tool having a tissue interface 120. The electrosurgical tool 102 can include a first tine 122A and a second tine 122B that collectively define a stimulating pathway 132 for delivery of the electrosurgical waveform to tissue captured between the first tine 122A and the second tine 122B.

The system 100 can include one or more sensors 160 for monitoring operation of the electrosurgical tool 102. In a further aspect, the electrosurgical tool 102 of the system 100 can provide fail-safe measures to reduce injury to surrounding tissues including a relief pathway 136 for selective diversion of current away from tissue. This can be especially useful for situations such as coagulum buildup along the electrosurgical tool 102 that can introduce high impedance at the stimulating pathway 132 and endanger surrounding tissue, reduce efficacy of coagulation activity, or damage the electrosurgical tool 102 if left unmitigated. The system 100 can include one or more impedance components 170 that can introduce and/or otherwise modify a relief pathway impedance at the relief pathway 136 and, in some examples, a stimulating pathway impedance at the stimulating pathway 132. Further, the system 100 can include a grounding configuration module 180 that can selectively configure and re-configure a grounding configuration of one or more contacts of the electrosurgical tool 102.

Additional features of the system 100 can include a user interface 190 that can provide operating information about the electrosurgical tool 102 to a practitioner. The user interface 190 can also, in some embodiments, receive inputs from the practitioner for adjusting parameters of the electrosurgical waveform. Example inputs can include toggling between one or more settings that adjust parameters of the electrosurgical waveform to fit specific needs, such as coagulation, cutting, and adjusting waveform parameters (e.g., shape, amplitude, frequency, crest factor) for specific types of tissue.

In some aspects, the waveform generator 140 can generate the electrosurgical waveform according to various waveform parameters. The electrosurgical waveform is not limited to a simple sine wave, square wave, etc., and can be generated as a combination (e.g., summation, multiplication, or another suitable combination method) of one or more individual waveforms. Combining the individual waveforms that result in the electrosurgical waveform exhibiting a unique shape, frequency distribution, amplitude variation, etc. that can be tailored to the specific electrosurgical applications.

Further, the waveform generator 140 can generate a first electrosurgical waveform and a second electrosurgical waveform, where the second electrosurgical waveform is phase-shifted by 180 degrees (π radians) relative to the first electrosurgical waveform. Simultaneous application of the first electrosurgical waveform and the second electrosurgical waveform to tissue can reduce unintended spread of electrical energy (e.g., electrical charge, voltage, current, etc.) to surrounding tissues. Other examples may apply phase-shifting by other amounts besides 180 degrees.

### Electrosurgical Waveforms for Electrosurgical Applications and Motivations

For electrosurgical applications, sinusoidal waveforms are usually applied in commercial products with frequencies in the 200 kHz to 1.2 MHz range. The output voltage generally varies from 100-1000 Vpp, depending on the desired surgical application. Many of these current era radiofrequency generators have adopted a sinusoidal waveform for delivery of energy. This sinusoidal waveform can then be modified to permit “off” periods within the stimulation waveform. Notably, off periods are points in time where no stimulus is being applied to the tissue (e.g., a “zero” or “neutral” voltage), resulting in a drop in efficiency of tool utilization. These off periods result in an increase in a term called the crest factor (CF), which usually describes a ratio between peak values and average values of a periodic signal over time. The CF can be considered a feature of electrosurgical waveforms that is expected to impact its likelihood for coagulation versus cutting (e.g., more effective for cutting when the crest factor is low, more effective for coagulation when the crest factor is high). CF can be defined as follows for a continuous sinusoidal waveform, where V_{MAX} indicates a maximum or peak voltage and V_{RMS} indicates a root mean square value of the waveform:

$$C F = {V}_{M AX} / {V}_{R MS}$$

For waveforms that are non-continuous, the CF can be determined as follows, where Vₚₚ indicates a peak-to-peak voltage of the waveform:

$$C F = {V}_{PP} / ( 2 {V}_{R MS} )$$

Given the consistency of a true sine wave, the CF is \sqrt{2} or approximately 1.4. This represents the optimized waveform for electrosurgical cutting applications. As the CF increases, through the delivery of a longer off percentage of the waveform, the electrosurgical result is a greater exhibition of coagulation rather than cutting. These settings are referred to as “blend” settings on commercial bipolar radiofrequency generators. As the off-percentage increases to the near maximum, >90% of the duty cycle, the CF reaches >10 and at this level the electrosurgical result is the waveform that is most appropriate for pure coagulation applications. This is a delicate balance given the greater the coagulation, the greater the local tissue damage at these higher CF values. Therefore, an ideal electrosurgical waveform for electrosurgical applications are those that deliver a sufficient CF to enable optimal and efficient coagulation while also limiting the local spread of current to unintended tissues.

As described herein and understood by one of skill in the art, injury to patients in surrounding tissues is a difficulty and continued challenge in performing microsurgical procedures and applications and related devices. There is a delicate balance given the greater the coagulation, the greater the local tissue damage at these higher CF values. As such, the system 100 disclosed herein is configured to deliver an electrosurgical waveform having a sufficient CF to enable optimal efficient coagulation while also limiting the local spread of current to unintended tissues. Further, given variations in tissue properties across many possible electrosurgical applications, it is expected that parameters of an optimal electrosurgical waveform may also vary. As such, the present disclosure outlines systems and methods for constructing and delivering electrosurgical waveforms of varying shape, amplitude, and/or frequency that can be tailored to specific electrosurgical applications (i.e. tissues or desired local effects).

As understood herein, the system 100 includes devices and methods for generating an electrosurgical waveform for electrosurgical tasks. Electrosurgical tasks as discussed herein encompasses those involved in electrosurgery and applied in the surgical space by the electrosurgical tool 102. For example, electrosurgical tasks can include, but are not limited to, cutting tissues and coagulating tissues (e.g., by application of the electrosurgical waveform). Importantly, the system 100 provides devices and methods for generating the electrosurgical waveform such that the electrosurgical waveform exhibits properties conducive to an electrosurgical task.

These properties can include CF of the electrosurgical waveform, where a high CF is generally accepted as being conducive to a “coagulating” electrosurgical task and where a low CF is generally accepted as being conducive to a “cutting” electrosurgical task. In some examples, a CF that is conducive to an electrosurgical task may be somewhere in between what is colloquially accepted as being “low” or “high”, ensuring a balance between cutting and coagulation tasks. Further, a CF that is conducive to an electrosurgical task may be dependent upon other factors such as operating conditions of the system 100, or dielectric tissue properties of targeted tissue and/or non-targeted tissue. As such, the system 100 aims to optimize properties of the electrosurgical waveform such as a CF to be conducive to an electrosurgical task while minimizing injury.

The properties of the electrosurgical waveform that are conducive to an electrosurgical task can also include an amplitude of the electrosurgical waveform and/or a peak-to-peak magnitude of the electrosurgical waveform. Amplitude and/or peak-to-peak magnitude can be affected by parameters of the electrosurgical waveform as generated at an output of the waveform generator, and can also be affected by other aspects of the system such as impedances, contact arrangements and grounding configurations associated with the electrosurgical tool 102. An amplitude and/or a peak-to-peak magnitude that is conducive for an electrosurgical task can depend upon the specific electrosurgical task, and can also depend on tissue properties. An amplitude and/or a peak-to-peak magnitude that is conducive for an electrosurgical task can also depend upon operating conditions of the system 100. For example, higher amplitudes correspond with more energy being delivered to tissue for cutting tasks, and can overcome impedances introduced through coagulum buildup and other factors, although would further propagate the buildup of coagulum. However, higher amplitudes can also be associated with damage to surrounding non-targeted tissues (i.e. off-target effects). As such, the system 100 aims to optimize properties of the electrosurgical waveform such as amplitude and/or peak-to-peak magnitude to be conducive to an electrosurgical task while minimizing injury. Given the goal of electrosurgery is often to cease bleeding that originates along an exposed surface, the necessary electrical energy being applied to that surface simply needs to close capillaries, arterioles, or venules that are present along that surface area and not destroy the deeper tissue below that exposed surface. Therefore, a delicate balance of energy delivery is necessary to accomplish the goal and preserve the functionality of the deeper tissue.

The properties of the electrosurgical waveform that are conducive to an electrosurgical task can also include a frequency of the electrosurgical waveform. A frequency of the electrosurgical waveform that is conducive for an electrosurgical task can depend upon the specific electrosurgical task and can also depend on tissue properties, including tissue properties of targeted tissue and non-targeted tissue. For example, higher frequency of the electrosurgical waveform is associated with a lower peak-to-peak voltage applied at the tissue but also greater permeation through tissue. As such, the system 100 aims to optimize properties of the electrosurgical waveform such as frequency to be conducive to an electrosurgical task while minimizing injury.

The properties of the electrosurgical waveform that are conducive to an electrosurgical task can also include a shape of the electrosurgical waveform, which can be related to CF, amplitude, peak-to-peak magnitude and frequency. A shape of the electrosurgical waveform that is conducive for an electrosurgical task and can depend upon the specific electrosurgical task and can also depend on tissue properties, including tissue properties of targeted tissue and non-targeted tissue. As such, the system 100 aims to optimize properties of the electrosurgical waveform such as shape to be conducive to an electrosurgical task while minimizing injury.

### Electrosurgical Tool

FIGS. 2A and 2B show an example embodiment of the electrosurgical tool 102 of the system 100. The electrosurgical tool 102 can include the first tine 122A and the second tine 122B for grasping tissue 10 at the tissue interface 120 collectively defined at the “tips” of the first tine 122A and the second tine 122B. As shown, the electrosurgical tool 102 can provide an ergonomic form factor for gripping within a hand of a practitioner and manipulation of the tissue 10, and can provide connection to other components of the system 100 (e.g., the processor 150 and the waveform generator 140 shown in FIG. 1). In some embodiments, the first tine 122A and the second tine 122B of the electrosurgical tool 102 can be tensioned. In operation, a practitioner can grip a volume of tissue to be cut and/or cauterized between the first tine 122A and the second tine 122B.

FIG. 2B shows an enlarged view of the tissue interface 120 of the electrosurgical tool 102. The first tine 122A of the electrosurgical tool 102 can include a first stimulating contact 134A of the stimulating pathway 132 that applies the first electrosurgical waveform. Likewise, the second tine 122B of the electrosurgical tool 102 can include a second stimulating contact 134B of the stimulating pathway 132 that simultaneously applies the second electrosurgical waveform.

In some examples, the electrosurgical tool 102 can include a first relief contact 138A and a second relief contact 138B of the relief pathway 136 for selective diversion of electrical energy (e.g., electrical charge, voltage, current, etc.) away from tissue. The first relief contact 138A can be along the first tine 122A and the second relief contact 138B can be along the second tine 122B. The first relief contact 138A and the second relief contact 138B can be in communication with the impedance component(s) 170 that establish a high impedance of the relief pathway 136 to encourage current flow through the stimulating pathway 132 by default. When a high impedance is detected or otherwise present at the stimulating pathway 132, such as in the presence of coagulum buildup, the relief pathway 136 can provide a lower-impedance path for current to flow to “ground” or “neutral”. In some examples, the impedance component(s) 170 can include fixed resistive components or other components that vary an impedance of the relief pathway 136 responsive to control signals and/or based on the impedance of the stimulating pathway 132. The contacts (e.g., the first stimulating contact 134A, the second stimulating contact 134B, the first relief contact 138A and the second relief contact 138B) can each be separated by an insulating material (e.g., insulating regions 139 in FIG. 2B).

In some examples, one or more contacts of the electrosurgical tool 102 can be activated or deactivated as needed. One strategy for deactivating a contact can include increasing an impedance associated with the contact to be significantly higher than impedances associated with “active” contacts (e.g., to prevent current from passing through the “deactivated” contact). One example is discussed herein in which contacts belonging to one tine of the electrosurgical tool 102 are deactivated to permit electrosurgical tasks involving surface treatment and avoid translating energy across tissue captured at the electrosurgical tool 102.

Further, “selective diversion of electrical energy” as defined herein is used to describe how the relief pathway 136 can be selectively configured to divert electrical energy away from tissue when appropriate. As interpreted herein, the relief pathway 136 does not necessarily provide a direct path to ground or neutral for energy leaving the stimulating pathway 132, as the energy leaving a stimulating contact first enters the tissue to facilitate an electrosurgical task. During normal operation of the relief pathway 136, at any point in time, the relief pathway 136 may be momentarily configured to divert any amount of energy away from tissue as needed. For example, if appropriate for the given electrosurgical task such as those requiring higher amounts of energy to be present within the tissue, the relief pathway 136 may be momentarily configured to selectively prevent diversion of energy away from tissue (e.g., through a high impedance associated with the relief pathway 136). In another example, if appropriate for the given electrosurgical task such as those requiring some amount of energy to be present within the tissue also requiring mitigation of excessive energy buildup, the relief pathway 136 may be momentarily configured to allow selective diversion of some energy away from tissue (e.g., through a moderate impedance associated with the relief pathway 136) while preventing complete diversion of energy away from the tissue. For electrosurgical tasks where it is desirable to minimize energy buildup in tissue as much as possible, the relief pathway 136 may be momentarily configured to allow selective diversion of a significant amount of energy away from tissue (e.g., through a low impedance associated with the relief pathway 136). As such, the relief pathway 136 may be momentarily configured and re-configured for selective diversion of energy away from tissue as needed and as appropriate for the electrosurgical task.

In some embodiments, the electrosurgical tool 102 can include one or more sensors 160 for monitoring operation of the electrosurgical tool 102. For example, measurements captured by one or more sensors 160 can include but are not limited to: a temperature at the tissue, an impedance of the stimulating pathway 132, a current through the relief pathway 136 and/or the stimulating pathway 132, and a mechanical force applied at or between the first tine 122A and/or the second tine 122B of the electrosurgical tool 102. Further, each respective contact can be sufficiently separated from other contacts by an insulating material to avoid shorting between respective contacts.

Further, in some embodiments, the electrosurgical tool 102 can include the user interface 190 (one example is shown in FIG. 2A) that includes one or more display elements 192 for displaying a status, mode and/or other information pertinent to operation of the system 100. In the example, one or more display elements 192 can be color-coded LEDs that indicate operating status, and other information to the practitioner. Alternatively, audible cues can be prompted by the status of the electrosurgical tool 102 for the purposes of defining the operating status. Other information communicable through the user interface 190 are also possible, such as: aspects of the electrosurgical waveform such as a crest factor value, impedance values associated with the stimulating pathway and/or the relief pathway, current values measured through the stimulating pathway and/or the relief pathway, a force value measured at the electrosurgical tool 102, a temperature of tissue measured at the electrosurgical tool 102, and/or an “error” state (or lack thereof). Other options for the display elements 192 can include, for example, high-resolution or low-resolution digital displays with or without backlighting, seven-segment displays, analog displays, etc.

Further, the user interface 190 can include one or more input elements 194 for receipt of a control input from a practitioner. In the example shown, the input elements 194 includes a toggle switch for selectively switching between parameters conducive for coagulation and cutting. Other types of input elements are also contemplated, such as individual buttons, a keyboard, an application running on another device in communication with the electrosurgical tool 102, a scroll wheel, etc. While this example of the user interface 190 is shown at the electrosurgical tool 102, aspects of the user interface 190 can also be provided at a display device in communication with the processor 150 and/or the waveform generator 140 to communicate operating information to a practitioner and to receive control inputs. Control inputs received at the user interface 190 can be communicated to the processor 150, the waveform generator 140, the impedance components 170, the grounding configuration module 180 or another component of the system 100. Control inputs can be applied to modify aspects including: parameters of the electrosurgical waveform, threshold values, impedances of the stimulating pathway or relief pathway, a grounding configuration, and/or a configuration of the tissue interface 120 of the electrosurgical tool 102.

In some examples, an electrosurgical tool of the system 100 can include more than two tines, and can further have different or varying role assignments for a plurality of contacts positioned along a tissue interface of each respective tine. For example, an electrosurgical tool of the system 100 can include one or more auxiliary relief tines (e.g., in addition to the first tine 122A and the second tine 122B) having a plurality of relief contacts. In some embodiments, the one or more auxiliary relief tines can be connected in a fixed position relative to the first tine 122A and the second tine 122B by fixed connection or can be independently moveable and reconfigurable within the surgical space. One advantage to having auxiliary relief tines in addition to the first tine 122A and the second tine 122B includes the ability to place auxiliary relief tines at different locations within the surgical space to divert charge away from non-targeted tissue.

### Generating Electrosurgical Waveforms

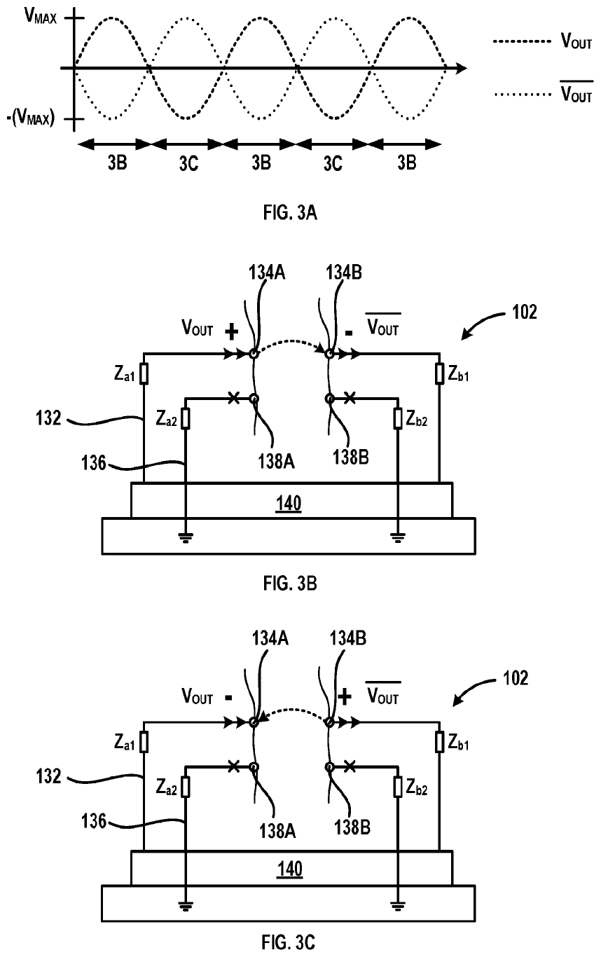

FIGS. 3A and 5A-7B show various example electrosurgical waveforms that can be generated by a waveform generator (e.g., waveform generator 140 of FIG. 1) and applied to tissue at an electrosurgical tool (e.g., electrosurgical tool 102 shown in FIGS. 2A and 2B). FIGS. 3B and 3C show example diagrams for generation of a phase-shifted electrosurgical waveform shown in FIG. 3A.

### 180-Degree Phase-Shifted Electrosurgical Waveforms

FIG. 3A shows a first electrosurgical waveform V_{OUT} being a simple sine wave that oscillates between a maximum voltage V_{MAX} and a minimum voltage V_{MIN}=−(V_{MAX}). A second electrosurgical waveform V̅_̅{̅O̅U̅T̅}̅ is a 180-degree phase-shifted version of the first electrosurgical waveform such that a total peak-to-peak magnitude of the electrosurgical waveform is two times the magnitude of the first electrosurgical waveform or the second electrosurgical waveform. FIGS. 3B and 3C are corresponding simplified diagrams that show direction of current being applied at periodic intervals. When the first electrosurgical waveform is applied to tissue, e.g., by the first stimulating contact 134A of the stimulating pathway 132, the second electrosurgical waveform is similarly applied to tissue with opposing polarity, e.g., by the second stimulating contact 134B of the stimulating pathway 132. As voltage is applied to tissue along one direction at the first stimulating contact 134A applying the first electrosurgical waveform, equal and opposing voltage is also applied to tissue along the opposite direction. FIG. 3B shows one example where a value of V_{OUT} is above “zero” and a corresponding value of V̅_̅{̅O̅U̅T̅}̅ is below “zero”, corresponding with intervals indicated by “3B” in FIG. 3A. As shown, when V_{OUT} is “high” and V̅_̅{̅O̅U̅T̅}̅ is “low”, current is drawn from the first stimulating contact 134A, through the tissue captured at the electrosurgical tool 102, and towards the second stimulating contact 134B. FIG. 3C shows one example where a value of V_{OUT} is below “zero” and a corresponding value of V̅_̅{̅O̅U̅T̅}̅ is above “zero”, corresponding with intervals indicated by “3C” in FIG. 3A. As shown, when V_{OUT} is “low” and V̅_̅{̅O̅U̅T̅}̅ is “high”, current is drawn in the opposite direction from the second stimulating contact 134B, through the tissue captured at the electrosurgical tool 102, and towards the first stimulating contact 134A. Colloquially, this arrangement can be viewed in such a way that the second electrosurgical waveform “cancels out” the first electrosurgical waveform to reduce propagation of energy to unintended areas, while increasing a total peak-to-peak magnitude of voltage applied to tissue captured at the electrosurgical tool 102. The phase shifting also permits a reduction in input volume magnitude while preserving the peak-to-peak magnitude of voltage experienced by the tissue through the “push-pull” arrangement between the contacts (i.e., stimulating contacts 134A and 134B). Note that while the example shown outlines a 180-degree phase shift, other amounts may also be advantageous for some electrosurgical applications. A first validation study discussed herein provides support for this concept.

### Example 1

### Waveform Optimization for Efficient and Safe Bipolar Application

1. 180-degree Phase-Shifted Waveform
2. Multisine (N=2)
3. Multisine (N>2)
4. Square wave summated with a sinusoidal wave
5. Sawtooth wave summated with a sinusoidal wave
6. Waveform resembling amplitude-modulated (AM) wave

A laboratory investigation of the concepts discussed above was undertaken using a bipolar electrocautery device, water bath, tap water, a waveform generator, and a digital acquisition device (DAQ). An experimental setup included the waveform generator connected to the bipolar electrocautery device such that the two channels of the bipolar device could be stimulated or serve as ground, as needed. The DAQ was connected to the measurement contacts spaced such that the voltage differential at the 1 cm to 2 cm distance interval from the source of stimulation was recorded.

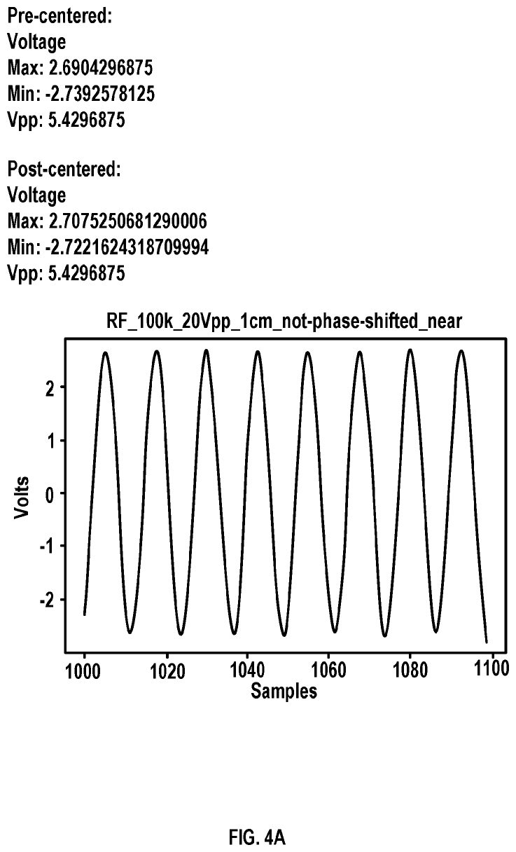

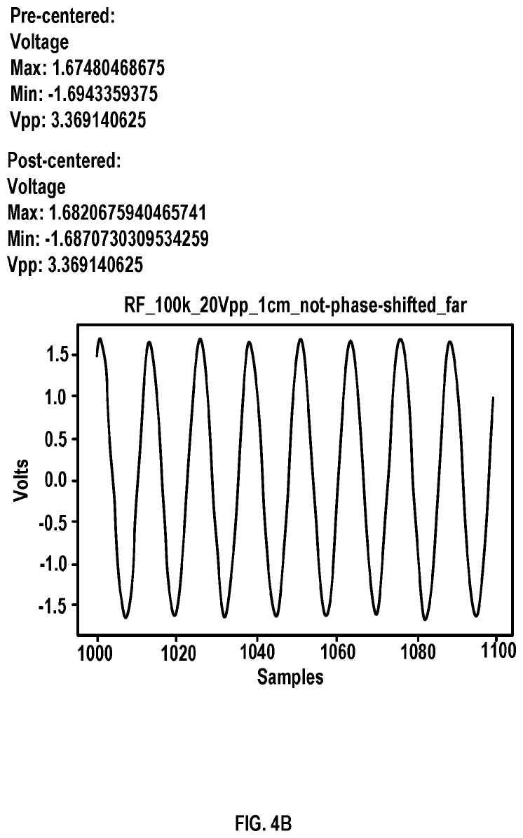

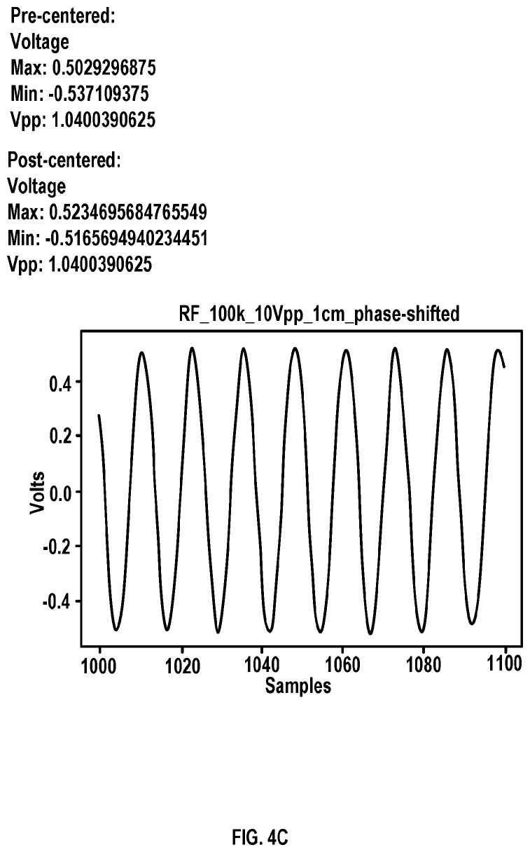

FIGS. 4A-4C show resulting measurement outputs following application of a 100 kHz sine waveform of a 20 VPP input. This 20 VPP input is necessary to provide a 20 VPP differential between the voltage peak and the ground contact (V=0). Due to there being a closer proximity of the left bipolar tip compared to the measurement contacts within the experimental setup, two graphs were generated at the DAQ: (1) where the stimulation source was “near” to the measurement contact (FIG. 4A); and (2) where the stimulation source was “far” from the measurement contact (FIG. 4B).

To demonstrate the value of a phase-shifted waveform to reduce the unintended spread of voltage to the surrounding tissue, a 100 kHz 180-degree phase-shifted waveform was delivered at 5 V (10 VPP) to the bipolar contacts. The resulting voltage measurement at distances of 1 cm and 2 cm from the stimulation source is reported in FIG. 4C.

As shown, a significant difference was observed in that the unintended spread of voltage throughout tissue as a result of bipolar electrocautery application is reduced by using a 180-degree phase-shifted waveform. Specifically, an 81% reduction in voltage within the 1-2 cm interval from the bipolar electrocautery device. Notably, this difference may change with varying degrees of separation between the bipolar contact tips (1 cm in this example).

### Optimizing Electrosurgical Waveforms for Electrosurgical Applications

While existing devices are aimed at generating waveforms that enable coagulation and cutting applications, the blend settings on these devices rely on “off” duty cycling to avoid the premature cutting of target tissue, e.g., by periodically pulling an output voltage to “zero” or “neutral” for a portion of the waveform. To enable the efficient conduction of coagulation and cutting, in one embodiment of the system 100, the waveform generator 140 can generate an electrosurgical waveform for application at the electrosurgical tool 102 that is a combination (e.g., by summation, product, or another suitable combination method) of a plurality of individual waveforms. In this section, the electrosurgical waveforms shown are examples of the first electrosurgical waveform applied at the first stimulating contact 134A. In some embodiments, the electrosurgical waveforms shown in this section can be phase-shifted by 180 degrees to create the second electrosurgical waveform applied at the second stimulating contact 134B, where the second electrosurgical waveform and the first electrosurgical waveform are simultaneously applied to tissue by the electrosurgical tool 102.

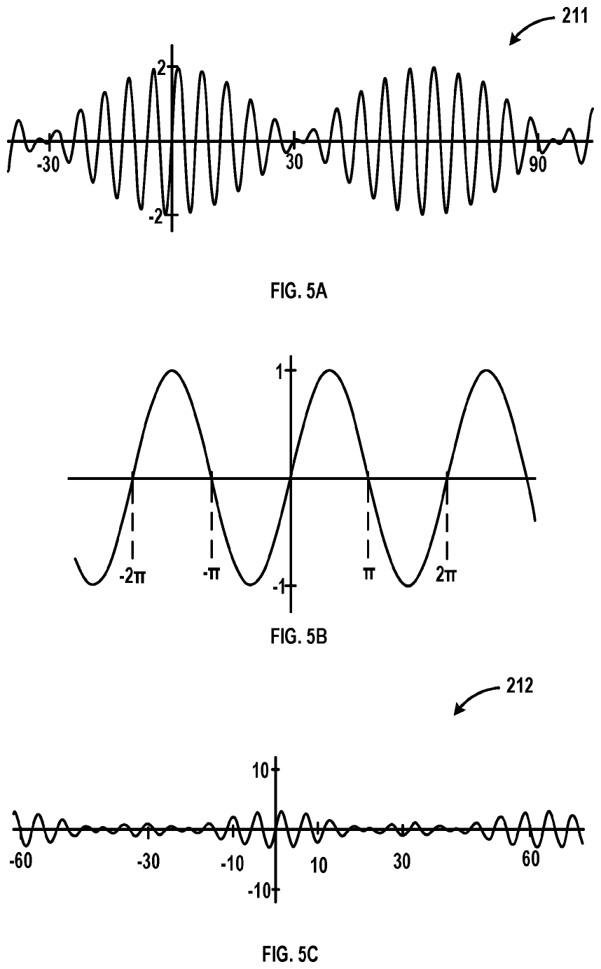

The waveform strategies demonstrated herein (FIGS. 5A, 5C, 6A-7B) are examples of electrosurgical waveforms that provide an innate interval within the electrosurgical waveform that a low amplitude of current/power is being delivered which could impart a lowered risk for injury to adjacent vulnerable tissue to the site of electrosurgery stimulation. This interval of lower energy stimulation can be optimal for cutting purposes while also avoiding the buildup of coagulum or eschar, which innately imparts a higher impedance barrier on stimulation between the contacts. In other words, electrosurgical waveforms that accomplish a reduction in peak energy during a portion of the electrosurgical waveform may provide a window for “cool-off” during the period and therefore avoid the buildup of coagulum. Further, the various electrosurgical waveforms can exhibit different shapes, amplitudes, and frequencies for different electrosurgical tasks. For example, increasing the time spent during “off periods” within an electrosurgical waveform can increase the CF of the electrosurgical waveform to be conducive for coagulation. In contrast, decreasing the time spent during “off periods” within an electrosurgical waveform can decrease the CF to be more conducive for cutting.

### Examples: Combination-Of-Sines

Sum of Two Sines: FIG. 5A shows an example electrosurgical waveform 211 in the form of a multisine waveform constructed from a summation of individual sine waves. In particular, the electrosurgical waveform 211 shown is a summation (vₒᵤₜ=v₁+v₂) of a first individual waveform (v₁=sin(t)) and a second individual waveform (v₂=sin(1.1 t)), where the first individual waveform has a first period and the second individual waveform has a second period that is greater than that of the first individual waveform. For comparison, the first individual waveform is shown in FIG. 5B. This example electrosurgical waveform 211 resembles an amplitude-modulated (AM) wave, where the first sine waveform can be considered superficially analogous to a carrier wave and the second sine waveform can be considered superficially analogous to a message wave that envelops the first sine waveform. One can observe the periodic amplitude drop relative to the root mean square of the wave. This distinguishes from other technologies that rely on introducing “off” periods (duty cycle modulation) where zero voltage is being applied. This electrosurgical waveform 211 is feasibly applicable for application in electrosurgery applications due to the “cool-down” period between stimulation maxima. While the example shown in FIG. 5A exhibits specific parameters, note that other variations of the electrosurgical waveform that follow a similar structure but have different parameters such as frequency, periodicity, amplitude, etc. are also contemplated. As such, the electrosurgical waveform applied by the electrosurgical tool 102 and waveform generator 140 of the system 100 is not limited to these specific examples. In some examples, the electrosurgical waveform can be constructed from two or more individual waveforms that can be phase-shifted relative to one another.

Sum of >2 Sines: FIG. 5C shows an example of an electrosurgical waveform 212 that can be constructed from more than two individual sine waves. In particular, this example is constructed from a summation (vₒᵤₜ=v₁+v₂+v₃) of a first sine waveform (v₁=sin(t)), a second sine waveform (v₂=sin(1.1 t)), and a third sine waveform (V₃=sin(1.2 t)). The first sine waveform can have a first period and the second sine waveform can have a second period that is greater than that of the first sine waveform. Further, the third sine waveform can have a third period that is greater than that of the second sine waveform. In this example, the electrosurgical waveform 212 is still periodic, but shows a more complex variation in amplitude and shape. This electrosurgical waveform 212 is feasibly applicable for application in electrosurgery applications due to the “cool-down” period between stimulation maxima. While the example shown in FIG. 5C exhibits specific parameters, other variations of the electrosurgical waveform are also contemplated that follow a similar structure but have different parameters, such as quantities of individual waveforms, frequency, periodicity, amplitude, etc. As such, the electrosurgical waveform applied by the electrosurgical tool 102 and waveform generator 140 of the system 100 is not limited to these specific examples.

Further, in some examples, the electrosurgical waveform can be constructed from two or more individual waveforms that can be phase-shifted relative to one another. Note that in this example, the phase-shifted individual waveforms are different from the first electrosurgical waveform and the second electrosurgical waveform being phase-shifted by 180-degrees. The phase-shifted individual waveforms can be combined to form the electrosurgical waveform, and the electrosurgical waveform can subsequently be used to generate the first electrosurgical waveform and the second electrosurgical waveform. As discussed, the second electrosurgical waveform is phase-shifted by 180-degrees relative to the first electrosurgical waveform.

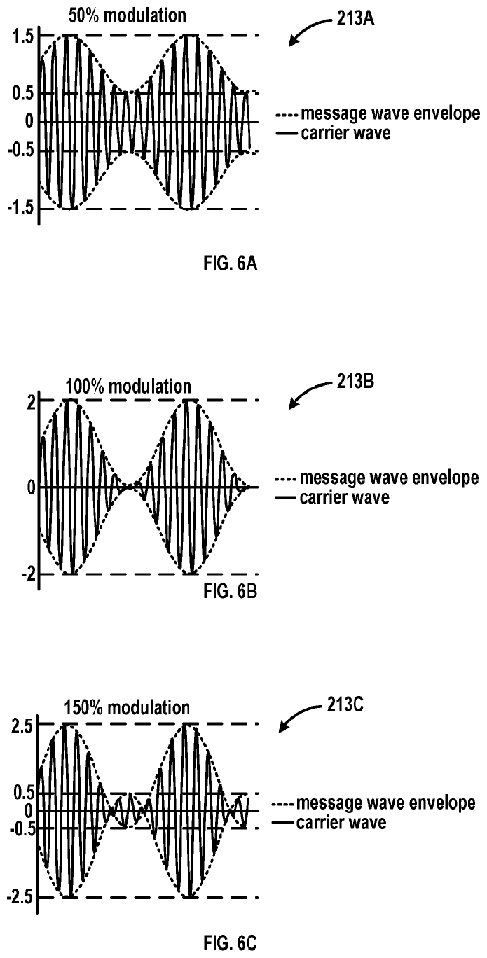

True Amplitude Modulation: FIGS. 6A-6C show various examples of electrosurgical waveforms 213A-213C that can be constructed using amplitude modulation with varying degrees of modulation depth (50%, 100%, and 150% respectively). In these examples, the modulation depth affects the amplitudes of each respective (AM-based) electrosurgical waveform 213A-213C, and the message waves that envelop respective carrier waves to are simple sinusoidal waveforms. This is in contrast with most AM waves in practical applications such as those involved in talk radio broadcasts where the message waves are usually aperiodic, such as those obtained by capturing a human voice. These electrosurgical waveforms 213A-213C are also examples of those feasibly applicable for application in electrosurgery applications due to the “cool-down” period between stimulation maxima. To construct the electrosurgical waveforms 213A-213C shown in FIGS. 6A-6C, the waveform generator 140 can apply an amplitude modulation operation to the individual waveforms (e.g., rather than a simple summation of the individual waveforms). Further, while the examples shown in FIGS. 6A-6C exhibit specific parameters, other variations of the electrosurgical waveforms are contemplated that follow a similar structure but have different parameters such as frequency, periodicity, amplitude, etc. As such, the electrosurgical waveforms applied by the electrosurgical tool 102 and waveform generator 140 of the system 100 are not limited to these specific examples. The electrosurgical waveforms 213A-213C demonstrated in FIGS. 6A-6C are different from that of FIG. 5A given the innate preservation of the individual sinusoidal waveforms within the electrosurgical waveforms 213A-213C, and can enable software modification of the individual waveforms for further modulation applications.

### Examples: Electrosurgical Waveforms Constructed from Different Shapes

In some examples, the shape and behavior of the electrosurgical waveform can affect its effectiveness for electrosurgical applications. In this section, various examples of electrosurgical waveforms constructed using individual waveforms of alternative shapes are provided. As more research is conducted in the electrosurgical space, practitioners can construct electrosurgical waveforms that exhibit alternative shapes that may be optimal for electrosurgery while reducing injury.

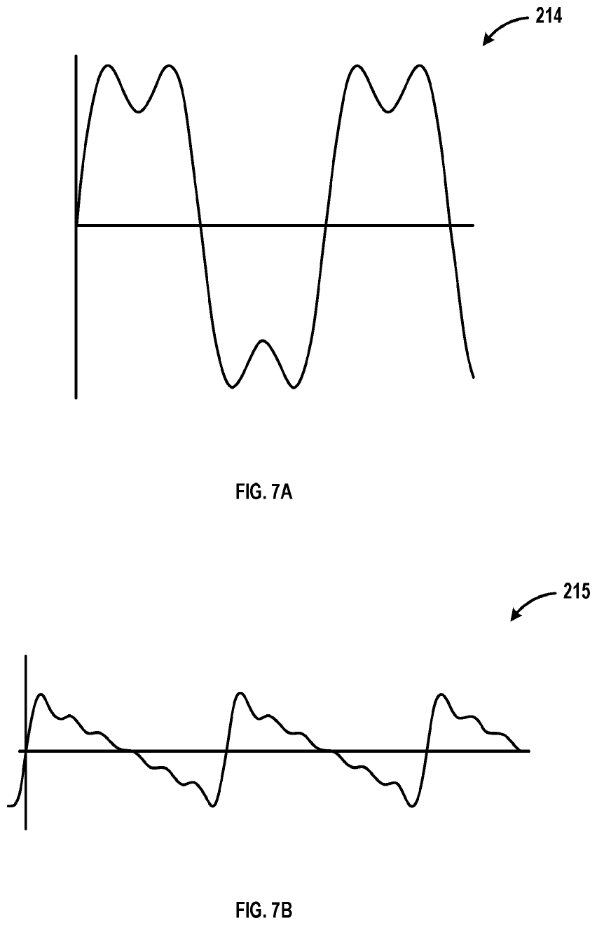

Square wave and Sine wave: FIG. 7A shows another example electrosurgical waveform 214 constructed from a summation of a square wave and a sine wave. Importantly, the electrosurgical waveform 214 shown in FIG. 7A also provides an innate interval of higher amplitude that can be conducive for cutting and an innate interval of lower amplitude that can be conducive for coagulation, where energy is still being delivered to tissue during the innate interval of lower amplitude. This is in opposition to previous technologies that provide an innate interval of zero amplitude. The periodicity and ratio of the innate intervals of lower amplitude and higher amplitude can be carefully controlled by selection of appropriate parameters of each individual waveform that, when combined, result in the electrosurgical waveform 214. Similarly, the frequency of the electrosurgical waveform 214, which can affect aspects such as how the electrosurgical waveform 214 permeates through tissue, can be carefully controlled by selection of appropriate parameters of each individual waveform. This electrosurgical waveform 214 is feasibly applicable for application in electrosurgery applications due to the “cool-down” period between stimulation maxima.

Sawtooth wave and Sine wave: FIG. 7B shows another example electrosurgical waveform 215 constructed from a summation of a sawtooth wave and a sine wave. Importantly, the electrosurgical waveform 215 shown in FIG. 7B also provides an innate interval of higher amplitude that can be conducive for cutting and an innate interval of lower amplitude that can be conducive for coagulation, where energy is still being delivered to tissue during the innate interval of lower amplitude. The periodicity and ratio of the innate interval of lower amplitude and higher amplitude can be carefully controlled by selection of appropriate parameters of each individual waveform that, when combined, result in the electrosurgical waveform 215. Similarly, the frequency of the electrosurgical waveform 215, which can affect aspects such as how the electrosurgical waveform 215 permeates through tissue, can be carefully controlled by selection of appropriate parameters of each individual waveform. This electrosurgical waveform 215 is feasibly applicable for application in electrosurgery applications due to the “cool-down” period between stimulation maxima.

While these examples shown in FIGS. 7A and 7B show electrosurgical waveforms constructed using a square wave or a sawtooth wave combined with sine waves, other electrosurgical waveforms are also contemplated that can be constructed from different shapes, such as a triangular wave. Further, other electrosurgical waveforms are also contemplated that can be constructed from more than two waveforms of varying shapes, such as a combination of a sine wave, a square wave, and a triangular wave. In a further aspect, principles of frequency modulation (FM) can also be applied when combining individual waveforms into an electrosurgical waveform to modulate frequencies of the electrosurgical waveform.

### Properties for Electrosurgical Applications

Importantly, the electrosurgical waveforms shown in FIGS. 5A, 5C, and 6A-7B provide innate intervals of higher amplitude that can be conducive for cutting and innate intervals of lower amplitude that can be conducive for coagulation, where energy is still being delivered to tissue during the innate interval of lower amplitude. This is in opposition to previous technologies that provide an innate interval of zero amplitude. The periodicity and ratio of the innate intervals of lower amplitude and higher amplitude can be carefully controlled by selection of appropriate parameters of each individual waveform. When combined, the individual waveforms result in the electrosurgical waveform. As such, the resultant CFs can be selected to be optimal for cutting and/or coagulation for the specific electrosurgical application.

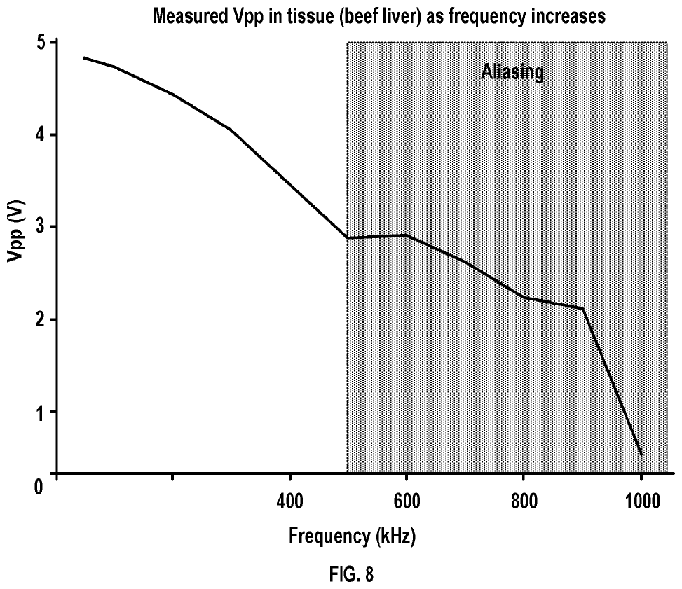

Frequency: Similarly, the frequency of the electrosurgical waveform can be carefully controlled by selection of appropriate parameters of each individual waveform. The frequency of the electrosurgical waveform can affect aspects such as how the electrosurgical waveform permeates through tissue. Another case study applied to beef liver is presented to examine the effect of frequency of an electrosurgical waveform on tissue. As shown in FIG. 8, as frequency of the electrosurgical waveform increases, permeation of voltage through tissue appears to decrease (although aliasing was observed during measurement when the frequency exceeded ˜500 kHz). As such, selection of parameters affecting frequency for the electrosurgical waveform can be determined based on properties of the tissue to ensure that the electrosurgical waveform does not permeate to unintended areas. Parameter selection could also be optimized for the desired depth of penetration within the tissue, such as surface area delivery versus deep tissue permeation.

Parameter Selection: Further, in some examples, the parameters of the electrosurgical waveform, including parameters of individual waveforms that combine to form the electrosurgical waveform, can be selected by the processor 150 and/or by the practitioner. The processor 150 can be operable for receiving input from the practitioner that indicates one or more aspects of the electrosurgical waveform to be produced, such as a target crest factor and shape. In some aspects, the processor 150 can “fill in the blanks” by determining one or more parameters that are needed to construct the electrosurgical waveform. In other aspects, the processor 150 can be operable for selecting parameters based on input from the practitioner that indicates a purpose of the electrosurgical waveform, such as a tissue type, a location of the tissue (including location of tissue relative to patient-specific imaging), prioritizing coagulation vs. cutting, minimizing permeation to surrounding tissues, etc. Additional testing and experimentation can be performed to identify what waveform shapes and parameters are best for different tissues and applications.

In some embodiments the processor 150 is operable to infer parameters of the electrosurgical waveform, including individual waveforms that combine to form the electrosurgical waveform. This may be achieved using numerical methods such as logistic regression or methods that incorporate machine learning. The CF and various other aspects of the electrosurgical waveform, including the individual waveforms that result in the electrosurgical waveform, can be calculated through suitable methods. For example, the processor 150 can be operable to determine parameters of the electrosurgical waveform using one or more approximation algorithms or functions.

In some examples, parameters associated with the electrosurgical waveform can be stored at a memory or other component in association with the waveform generator 140 and/or the processor 150 as a setting of a plurality of settings that may be retrieved and applied to generate the electrosurgical waveform. Further, parameters associated with the electrosurgical waveform can be modified with input from an authorized individual at any time during an electrosurgical procedure—in other examples, modification of parameters associated with the electrosurgical waveform can require authorization before application (e.g., to prevent accidental or intentional tampering by an unauthorized individual).

### Monitoring Feedback and Adjusting Accordingly

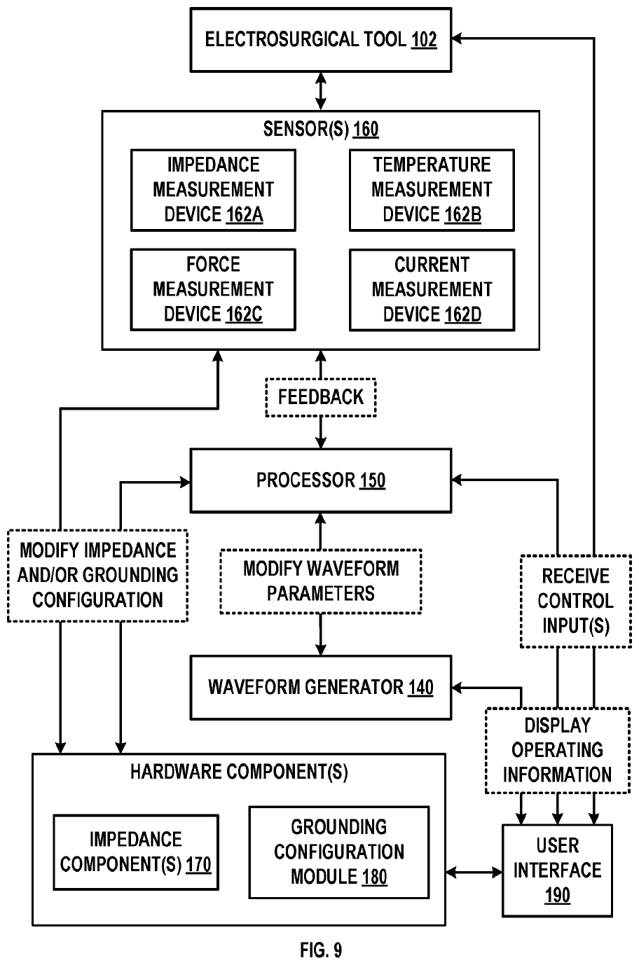

In some examples, with reference to FIG. 9, the system 100 can monitor operation through feedback measured at or otherwise in association with the electrosurgical tool 102 and adjust to ensure optimized delivery of the electrosurgical waveform. Feedback measured at the electrosurgical tool 102 can be captured through more than one modality, accomplished through one or more sensors 160 in association with the electrosurgical tool 102.

In some embodiments the processor 150 of the system 100 can receive feedback from the sensors 160, and can determine and apply one or more responses based on the feedback, including feedback from a combination of two or more modalities. Responses can include communicating with the waveform generator 140 to modify one or more electrosurgical waveform parameters and/or communicating with the impedance component(s) 170 to modify an impedance of the stimulating pathway 132 and/or the relief pathway 136. Electrosurgical waveform parameters that can be modified can include, but are not limited to: shape, amplitude, frequency, crest factor, RMS value, quantities of individual waveforms used to generate the electrosurgical waveform, and/or combinations or perturbations of the individual waveforms. Other parameters of the system 100 that can be modified can include, but are not limited to: a grounding configuration of one or more contacts, role assignments of one or more contacts (e.g., sensing, applying the stimulating waveform, diverting energy away from tissue), activation or deactivation state of one or more contacts, and values of impedances associated with the stimulating pathway 132 and/or the relief pathway 136.

The system 100 can also include one or more hardware components in communication with the electrosurgical tool 102 that can apply one or more responses based on the feedback. Hardware components can include the impedance component(s) 170 and the grounding configuration module 180. In some examples, the hardware components can incorporate measured feedback. Responses that can be applied by the hardware components based on the measured feedback can include actions such as: adjusting a relief pathway impedance of the relief pathway 136 by the impedance component(s) 170 based on an impedance at the stimulating pathway 132 and without additional input from the processor 150, and updating a grounding configuration of the electrosurgical tool 102. In other embodiments, the hardware components can receive control signals from the processor 150 and/or the waveform generator 140 and apply one or more responses based on the control signals.

In some embodiments the system 100 can include the user interface 190 that can provide operating information about, for example, a status or mode of the system, feedback received and/or otherwise interpreted, and/or responses being taken by the system 100. The user interface 190 can also receive control inputs to adjust one or more modes or parameters of the system 100.

### Example 2

### Feedback on an Optimized Electrosurgery Waveform

1. Impedance measurement
   a. Modulation of electrosurgical waveform parameters (such as those mentioned above)
   b. Modulation of impedance at stimulating pathway and/or relief pathway
2. Temperature measurement
   a. Cold temperature threshold—modulation of voltage, waveform (those mentioned above), impedance values, and/or ground designation
   b. Hot temperature threshold—modulation of voltage, waveform (those mentioned above), impedance values, and/or ground designation
3. Force/Strain on device
   a. Force measurement device used to determine the active circuit configuration, impedance values, and/or ground designation
4. Current through relief pathway and/or stimulating pathway
   a. Modulation of electrosurgical waveform parameters (such as those mentioned above)
   b. Modulation of impedance at stimulating pathway and/or relief pathway

Impedance feedback: Impedance measurement and modulation of a multitude of parameters for electrosurgical applications is feasible, and has not been described within the context of an electrosurgical waveform constructed by combination of a plurality of individual waveforms. Therefore, in some embodiments, impedance feedback can be adopted for modulation of the delivered sum-of-sines waveform such that modification to one or multiple of the composing sinusoid waveforms could be altered in response to impedance increases or decreases. These parameters for modulation can include but are not limited to: input voltage/current (i.e. amplitude), waveform frequency, duty cycle, grounding configuration, or periodicity.

For example, in some embodiments, the system 100 can adopt impedance feedback for modulation of the electrosurgical waveform in response to increase or decrease of impedance measured at the stimulating pathway 132 of the electrosurgical tool 102. In some embodiments, the sensors 160 of the system 100 can include an impedance measurement device 162A in association with the stimulating pathway 132 and, in some embodiments, the relief pathway 136. Responses of the system 100 to impedance measured at the stimulating pathway 132 can include modifying parameters that dictate construction of the electrosurgical waveform such as: input voltage/current or amplitude, waveform frequency, duty cycle, grounding configuration, and periodicity. Other responses of the system 100 to impedance measured at the stimulating pathway 132 can include increasing or decreasing an impedance of the relief pathway 136 and/or the stimulating pathway 132 to encourage or discourage current diversion through the relief pathway 136, e.g., by the impedance component(s) 170 discussed in greater detail herein.

Temperature Feedback: In another aspect, the system 100 can adopt temperature feedback for modulation of the electrosurgical waveform or impedance at the stimulating pathway 132 and/or relief pathway 136 in response to increase or decrease of temperature (e.g., relative to a hot threshold value or a cold threshold value) measured at or around tissue captured at the electrosurgical tool 102. The sensors 160 of the system 100 can include a temperature measurement device 162B such as a thermal probe at the electrosurgical tool 102. In some examples, the electrosurgical tool 102 can include dual-function contacts, e.g., where the first stimulating contact 134A, the second stimulating contact 134B, the first relief contact 138A, and/or the second relief contact 138B are also operable for thermal sensing. Responses of the system 100 to temperature measured at or around tissue captured at the electrosurgical tool 102 can include modifying parameters that dictate construction of the electrosurgical waveform such as: input voltage/current or amplitude, waveform frequency, duty cycle, grounding configuration, and periodicity. Other responses of the system 100 to temperature measured at or around tissue captured at the electrosurgical tool 102 can include increasing or decreasing an impedance of the relief pathway 136 and/or the stimulating pathway 132 to encourage or discourage current diversion through the relief pathway 136, e.g., by the impedance components 170 discussed in greater detail herein.

Force or Strain Feedback: Further, the system 100 can adopt force or strain feedback for modulation of the electrosurgical waveform in response to an increase or decrease in force or strain measured at the electrosurgical tool 102. The sensors 160 of the system 100 can include a force measurement device 162C such as a tensometer or strain gauge at the electrosurgical tool 102. Responses of the system 100 to force or strain measured at the electrosurgical tool 102 can include modifying parameters that dictate construction of the electrosurgical waveform such as: input voltage/current or amplitude, waveform frequency, duty cycle, grounding configuration, and periodicity. Other responses of the system 100 to force or strain measured at the electrosurgical tool 102 can include increasing or decreasing an impedance of the relief pathway 136 and/or the stimulating pathway 132 to encourage or discourage current diversion through the relief pathway 136, e.g., by the impedance components 170 discussed in greater detail herein. In one example, the system 100 can adjust waveform parameters and/or impedance values to increase a voltage/current applied to tissue when the practitioner applies a greater force to the first tine 122A and/or the second tine 122B of the electrosurgical tool 102. This concept arises from a sense that it is unlikely that the practitioner would be manipulating micro-surgically relevant anatomy with greater force application to the electrosurgical tool 102. Further, if the voltage or current applied in the form of the electrosurgical waveform is too “low” for the specific electrosurgical task then the practitioner would intuitively apply greater force to the electrosurgical tool 102.

Current Feedback: In some embodiments, the system 100 can also monitor a current through the stimulating pathway 132 and/or relief pathway 136. The sensors 160 of the system 100 can include a current measurement device 162D. Based on the current, the system can apply responses such as modulating parameters of the electrosurgical waveform or impedance at the stimulating pathway 132 and/or relief pathway 136. Responses can be applied based on an increase or decrease of current measured through the stimulating pathway 132 and/or relief pathway 136. Responses of the system 100 to current measured through the stimulating pathway 132 and/or relief pathway 136 can include modifying parameters that dictate construction of the electrosurgical waveform. These parameters can include input voltage/current or amplitude, waveform frequency, duty cycle, grounding configuration, and periodicity. Other responses of the system 100 to current measured through the stimulating pathway 132 and/or relief pathway 136 can include increasing or decreasing an impedance of the relief pathway 136 and/or the stimulating pathway 132 to encourage or discourage current diversion through the relief pathway 136, e.g., by the impedance components 170 discussed in greater detail herein.

Responses applied by the system 100 can include mitigative actions that divert current away from the stimulating pathway 132, e.g., to avoid damage to surrounding tissues and/or the electrosurgical tool 102. For example, the system can receive of feedback indicating an excessively high current through the stimulating pathway 132 and an excessively high temperature at tissue as measured at or near the electrosurgical tool 102. In response, the system 100 can apply one or more responses that divert current away from the stimulating pathway 132 such as: increasing an impedance at the stimulating pathway 132 or decreasing an impedance at the relief pathway 136. Other possible responses can include modifying one or more parameters of the electrosurgical waveform or the individual waveforms associated with the electrosurgical waveform, terminating the electrosurgical waveform, or otherwise reducing or preventing application of voltage or current to tissues through the electrosurgical tool 102.

Responses applied by the system 100 based on received feedback can also include optimization actions such as updating one or more parameters of the electrosurgical waveform for application at the electrosurgical tool 102 to improve effectiveness. These responses can include: increasing a peak amplitude or decreasing an overall crest factor of the electrosurgical waveform to one that is more conducive for cutting tasks, increasing an overall crest factor of the electrosurgical waveform to one that is more conducive for cutting tasks, and/or updating one or more parameters of the electrosurgical waveform to increase or reduce a resultant area of effect within tissue.

In some examples, responses applied by the system 100 can be determined and applied based on properties of tissue at or around the electrosurgical tool 102. The system 100 can be operable for estimating tissue properties based on feedback received from one or more sensors 160 and for incorporating the tissue properties into the response. Responses can include adjusting one or more parameters of the electrosurgical waveform or adjusting an impedance of the stimulating pathway 132 and/or relief pathway 136 to increase effectiveness of the electrosurgical tool 102 with respect to the tissue type and reduce or prevent injury to surrounding tissues.

Further, responses applied by the system 100 can include generating one or more alerts for display at the user interface 190 (FIG. 2A). Responses applied by the system 100 can also include actions taken responsive to one or more control inputs received at the user interface 190.

### Stimulating and Relief Pathways

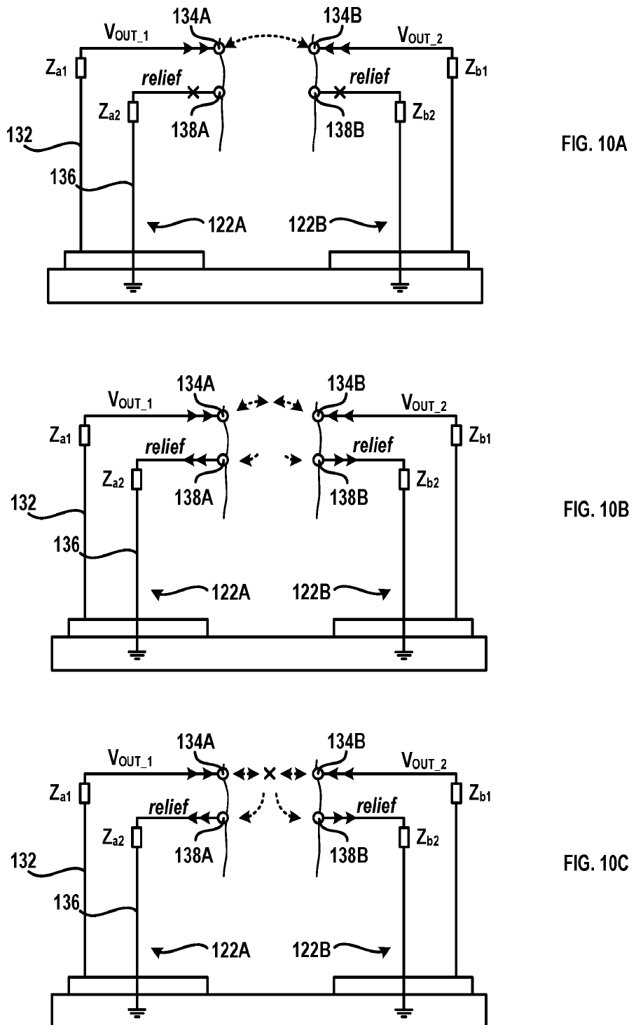

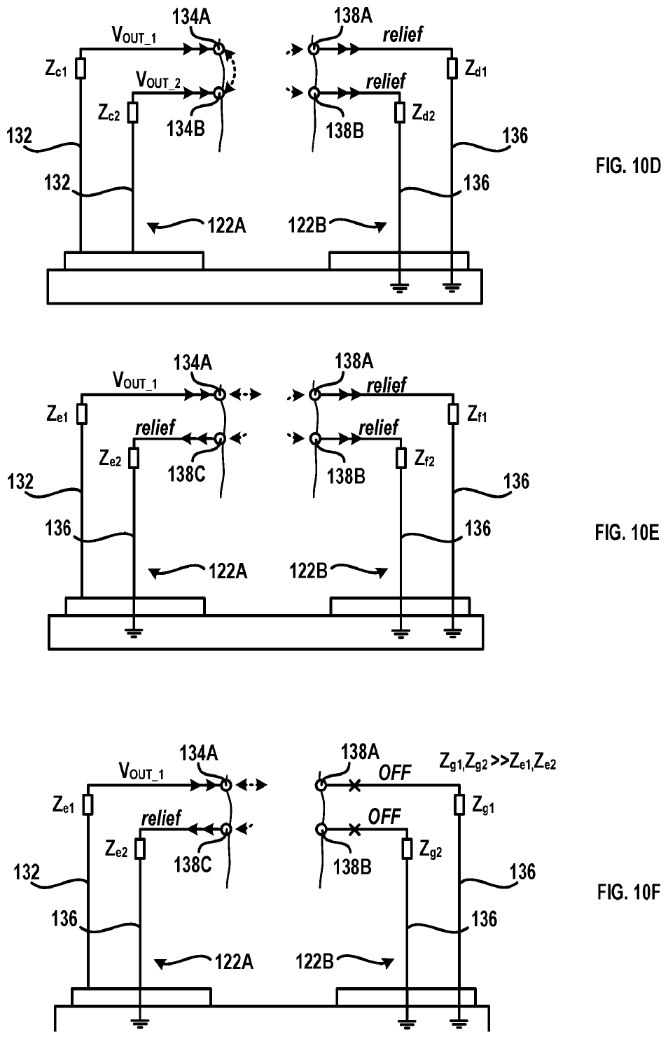

FIGS. 10A-10F are a series of simplified schematic diagrams showing the stimulating pathway 132 and the relief pathway 136 of the electrosurgical tool 102 as applied to tissue. As shown, the stimulating pathway 132 includes the first stimulating contact 134A along the first tine 122A and the second stimulating contact 134B along the second tine 122B for delivery of an electrosurgical waveform to tissue. In some embodiments, as will be described in greater detail herein, the first stimulating contact 134A can apply a first electrosurgical waveform denoted by V_{OUT_1} and the second stimulating contact 134B can apply a second electrosurgical waveform denoted by V_{OUT_2}. The second electrosurgical waveform V_{OUT_2} can be any suitable waveform. For example, the second electrosurgical waveform V_{OUT_2} can be identical to the first electrosurgical waveform V_{OUT_1}, or can be a phase-shifted version of the first electrosurgical waveform V_{out_1} (e.g., by 180 degrees or another value). In other examples, the parameters of the second electrosurgical waveform V_{OUT_2} can be different from the first electrosurgical waveform V_{OUT_1} to exhibit an alternative or complimentary shape, amplitude, peak-to-peak magnitude, CF, etc. Further, in some embodiments, the second stimulating contact 134B can apply a zero voltage, a positive DC voltage, or a negative DC voltage. In the examples shown, the first tine 122A and/or second tine 122B respectively include one or more sensors 160 that measure operational aspects of the system 100 and surgical space such as temperature of tissue and mechanical force applied at the electrosurgical tool 102. Further, as shown, the relief pathway 136 can provide a path to neutral (e.g., ground) for current that proceeds through a higher-impedance pathway.

In some examples, with reference to FIG. 10D, the first stimulating contact 134A and the second stimulating contact 134B of the stimulating pathway 132 can be positioned along the same tine (e.g., the first tine 122A or the second tine 122B). Likewise, the first relief contact 138A and the second relief contact 138B of the relief pathway 136 can be positioned along the same tine (e.g., the first tine 122A or the second tine 122B). FIG. 10E shows an alternative example with a single contact being assigned as the first stimulating contact 134A of the stimulating pathway 132, and with remaining contacts being assigned as the first relief contact 138A, the second relief contact 138B, and a third relief contact 138C of the relief pathway 136. Further, FIG. 10F shows an example similar to FIG. 10E, where the first relief contact 138A the second relief contact 138B along one of the tines (e.g., the second tine 122B) are deactivated.

### Illustrative Examples

FIG. 10A shows an example configuration of the electrosurgical tool 102 where there is little to no excessive impedance between the first stimulating contact 134A and the second stimulating contact 134B such that the stimulating pathway 132 is open and clear. In this example, the first electrosurgical waveform denoted by V_{OUT_1} and the second electrosurgical waveform denoted by V_{OUT_2} (if applicable) are applied to tissue captured at the electrosurgical tool 102. Further, the first relief pathway impedance Zₐ₂ and the second relief pathway impedance Z_{b2} are each greater than impedances associated with the stimulating pathway 132 including impedances introduced by the tissue. As such, due to high impedance at the relief pathway 136, electrical current is forced through the stimulating pathway 132.

FIG. 10B shows an example configuration of the electrosurgical tool 102 where there is increased impedance between the first stimulating contact 134A and the second stimulating contact 134B. Increased impedance at the stimulating pathway 132 can happen for many reasons, such as a change in tissue type and/or coagulum buildup that, if unmitigated, can reduce the effectiveness of the electrosurgical tool 102 and potentially cause damage to surrounding tissues. In this example, the first electrosurgical waveform denoted by V_{OUT_1} and the second electrosurgical waveform denoted by V_{OUT_2} (if applicable) are applied to tissue captured at the electrosurgical tool 102, however, the impedance at the stimulating pathway 132 may be closer to the values of the first relief pathway impedance Zₐ₂ and the second relief pathway impedance Z_{b2}. As such, some current may proceed through the (higher-impedance) relief pathway 136 to ground as shown.

FIG. 10C shows an example configuration of the electrosurgical tool 102 where there is excessive impedance between the first stimulating contact 134A and the second stimulating contact 134B. In this example, the impedance at the stimulating pathway 132 may be greater than the impedance at the relief pathway 136 (as defined by the values of the first relief pathway impedance Zₐ₂ and the second relief pathway impedance Z_{b2}). As such, the first electrosurgical waveform denoted by V_{OUT_1} and the second electrosurgical waveform denoted by V_{OUT_2} (if applicable) are diverted away from the tissue captured at the electrosurgical tool 102 and current proceeds through the (higher-impedance) relief pathway 136 to ground as shown.

FIG. 10D shows the first stimulating contact 134A and the second stimulating contact 134B being positioned along the same tine (e.g., the first tine 122A or the second tine 122B). Likewise, the first relief contact 138A and the second relief contact 138B are shown positioned along the same tine (e.g., the first tine 122A or the second tine 122B). In the example of FIG. 10D, the stimulating pathway 132 can be associated with the first tine 122A (or the second tine 122B) for application of the first electrosurgical waveform V_{OUT_1} and the second electrosurgical waveform V_{OUT_2} (if applicable). The relief pathway 136 can be associated with the second tine 122B (or the first tine 122A) as shown for directing excessive electrical charge away from the tissue. Impedances associated with the stimulating pathway 132 are denoted in FIG. 10D as a first stimulating pathway impedance Z_{c1} and a second stimulating pathway impedance Z_{c2}, belonging to the first tine 122A. Likewise, impedances associated with the relief pathway 136 are denoted in FIG. 10D as a first relief pathway impedance Z_{d1} and a second relief pathway impedance Z_{d2}, belonging to the second tine 122B.

FIG. 10E shows an alternative example with a single contact being assigned as the first stimulating contact 134A, and with remaining contacts being assigned as the first relief contact 138A, the second relief contact 138B, and a third relief contact 138C. In this example, the first stimulating contact 134A and the third relief contact 138C can be positioned along the same tine (e.g., the first tine 122A or the second tine 122B). In the example of FIG. 10E, the stimulating pathway 132 can be associated with one contact of the first tine 122A (or the second tine 122B) for application of the first electrosurgical waveform V_{OUT_1}. The relief pathway 136 can be associated with the second tine 122B (or the first tine 122A) and with the remaining contact of the first tine 122A as shown for directing excessive electrical charge away from the tissue. Impedances associated with the stimulating pathway 132 are denoted in FIG. 10E as a first stimulating pathway impedance Zₑ₁, belonging to the first tine 122A. Likewise, impedances associated with the relief pathway 136 are denoted in FIG. 10E as a first relief pathway impedance Zn and a second relief pathway impedance Z_{f2}, belonging to the second tine 122B, and a third relief pathway impedance Zₑ₂.

FIG. 10F shows an alternative example similar to the example of FIG. 10E, with a single contact being assigned as the first stimulating contact 134A, and with remaining contacts being assigned as the first relief contact 138A, the second relief contact 138B, and a third relief contact 138C. Similarly, the first stimulating contact 134A and the third relief contact 138C can be positioned along the same tine (e.g., the first tine 122A or the second tine 122B). In the example of FIG. 10F, the stimulating pathway 132 can be associated with one contact of the first tine 122A (or the second tine 122B) for application of the first electrosurgical waveform V_{OUT_1}. The relief pathway 136 can be associated with the remaining contact of the first tine 122A as shown for directing excessive electrical charge away from the tissue.

In contrast with the example of FIG. 10E, the first relief contact 138A and the second relief contact 138B along the second tine 122B are functionally deactivated or turned “off”. This can be implemented, for example, by assigning very high impedances to the first relief contact 138A and the second relief contact 138B to prevent current flow from the tissue and/or the first stimulating contact 134A from entering the relief pathway 136 at the first relief contact 138A or the second relief contact 138B. As such, impedances associated with the stimulating pathway 132 are denoted in FIG. 10F as a first stimulating pathway impedance Zₑ₁, belonging to the first tine 122A. An impedance associated with the third relief contact 138C of the relief pathway 136 is denoted in FIG. 10F as Zₑ₂. Impedances associated with the first relief contact 138A and the second relief contact 138B along the second tine 122B are respectively denoted by Z_{g1} and Z_{g2}, where Z_{g1}, Z_{g2} are significantly larger than Zₑ₁, Zₑ₂ (Z_{g1}, Z_{g2}>>Zₑ₁, Zₑ₂). This prevents current from flowing into the second tine 122B which can be optimal for some electrosurgical tasks by avoiding translating energy across tissue captured between the tines of the electrosurgical tool. To activate the first relief contact 138A and the second relief contact 138B (and/or to activate and re-assign their respective roles), values of impedances respectively associated with each can be lowered to enable current flow.

For example, the arrangement shown in FIG. 10F can enable electrical connection between the first stimulating contact 134A and the third relief contact 138C to permit surface treatment while minimizing penetration of energy into tissue. In some examples, activation or deactivation of selective contacts as outlined with respect to FIG. 10F can be performed over time in an alternating fashion (e.g., activating contacts associated with the first tine 122A while deactivating contacts associated with the second tine 122B, and vice versa) to avoid sinking current into the opposing tine.

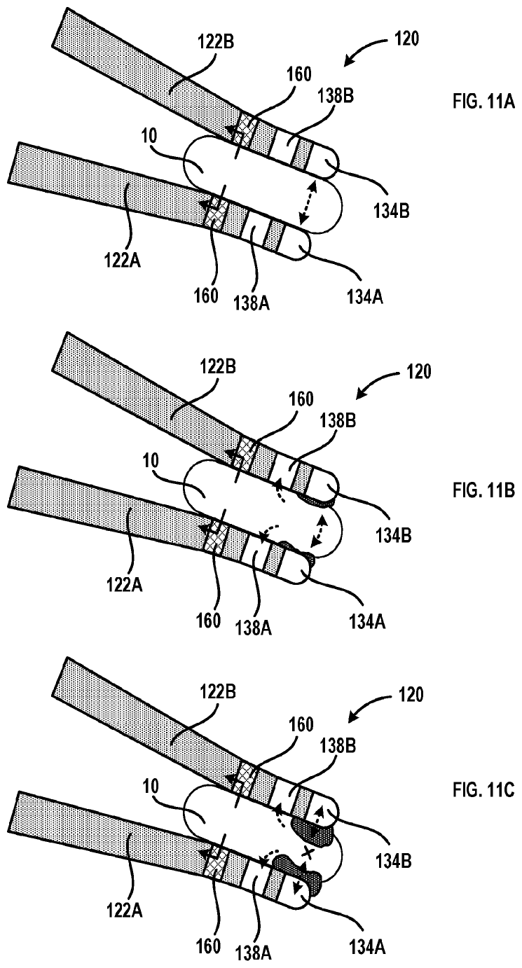

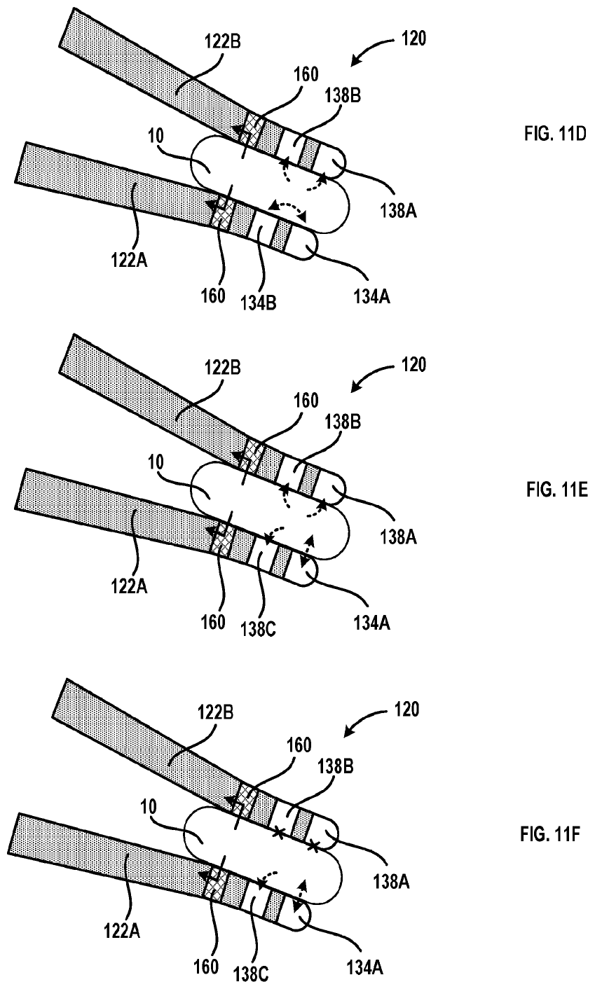

FIGS. 11A-11F correspond with the diagrams of FIGS. 10A-10F and show examples of the tissue interface 120 of the electrosurgical tool 102 with one or more contacts of the tissue interface 120 having various degrees of coagulum buildup. Coagulum buildup can affect the stimulating pathway impedance of the stimulating pathway 132 shown in FIGS. 10A-10C. FIGS. 11D-11F respectively correspond with the diagrams of FIGS. 10D-10F showing alternative assignments of the contacts of the tissue interface 120. FIGS. 11A-11F also show measurement of one or more properties of the tissue captured at the electrosurgical tool 102 by the sensors 160

As shown, in FIG. 11A, there is little to no coagulum buildup present along the first stimulating contact 134A and second stimulating contact 134B of the tissue interface 120. As such, the stimulating pathway 132 and the relief pathway 136 of the electrosurgical tool 102 can operate as shown in FIG. 10A in which the relief pathway 136 is the “higher-impedance” pathway. As a result, little to no electrical charge is diverted through the first relief contact 138A and the second relief contact 138B.

In FIG. 11B, there is some coagulum buildup present along the first stimulating contact 134A and second stimulating contact 134B of the tissue interface 120. As such, the stimulating pathway 132 and the relief pathway 136 can operate as shown in FIG. 10B in which there is some extra impedance at the stimulating pathway 132. As a result, some electrical charge from the tissue and/or applied at the first stimulating contact 134A and the second stimulating contact 134B may divert into the relief pathway 136 at the first relief contact 138A and the second relief contact 138B and will flow to ground or neutral.

In FIG. 11C, there is excessive coagulum buildup present along the first stimulating contact 134A and the second stimulating contact 134B of the tissue interface 120. Therefore, the stimulating pathway 132 and the relief pathway 136 of the electrosurgical tool 102 can operate as shown in FIG. 10C in which there is excessive impedance at the stimulating pathway 132. As a result, electrical charge from the tissue and/or applied at the first stimulating contact 134A and the second stimulating contact 134B diverts into the relief pathway 136 at the first relief contact 138A and the second relief contact 138B and will flow to ground or neutral.

FIG. 11D corresponds with FIG. 10D in which the first stimulating contact 134A and the second stimulating contact 134B of the stimulating pathway belong to the first tine 122A for application of the electrosurgical waveform to tissue 10 at the tissue interface 120. Likewise, the first relief contact 138A and the second relief contact 138B of the relief pathway belong to the second tine 122B for diversion of excessive charge away from tissue 10.

FIG. 11E corresponds with FIG. 10E in which the first stimulating contact 134A of the stimulating pathway belongs to the first tine 122A for application of the electrosurgical waveform to tissue 10 at the tissue interface 120. The first relief contact 138A and the second relief contact 138B of the relief pathway belong to the second tine 122B, and the third relief contact 138C of the relief pathway belongs to the first tine 122A for diversion of excessive charge away from tissue 10.

FIG. 11F corresponds with FIG. 10F in which the first stimulating contact 134A of the stimulating pathway belongs to the first tine 122A for application of the electrosurgical waveform to tissue 10 at the tissue interface 120. The first relief contact 138A and the second relief contact 138B of the relief pathway belong to the second tine 122B, and the third relief contact 138C of the relief pathway belongs to the first tine 122A for diversion of excessive charge away from tissue 10. The first relief contact 138A and the second relief contact 138B of the second tine are deactivated (e.g., through high impedance, as shown in FIG. 10F) to avoid transmitting energy across tissue captured between the first tine 122A and the second tine 122B.

### Example 3

### Bipolar Hardware Design for Accessory Ground

1. Multi-contact
   a. Contact number can include: 2, 3, 4, 5, 6, 7, 8 and so on.
   b. Accessory circuit opens depending on feedback measurements
   c. Accessory circuit continuously or intermittently open
2. Impedance variation between contact groups
3. Variation in active vs ground orientations
   a. 1 active, 3 ground (or other higher order configurations to match 1a contact count)
4. Variation in impedance of individual contacts
5. Contact shape/design

Electrosurgery devices to date are constructed as a simple bipolar circuit, however, as shown in FIGS. 10A-12, the system 100 can include one or more relief contacts (e.g., the first relief contact 138A and the second relief contact 138B) for selective diversion of current through the relief pathway 136. The relief pathway 136 connects to a ground or neutral voltage, e.g., through a higher-impedance pathway. In some examples, the relief pathway 136 is of a higher default impedance than the stimulating pathway 132. However, in the event of high impedance at the stimulating pathway 132, such as through coagulum buildup, the relief pathway 136 can provide a “path of lesser resistance” to ground or neutral. As such, one or more relief contacts of the relief pathway 136 provide an alternative current path to avoid the gradually higher impedance path developing at the stimulating pathway 132 through the buildup of coagulant/eschar. The relief pathway 136 can serve as a means of directing current/voltage away from tissue distal to the first tine 122A and second tine 122B of the electrosurgical tool 102. This relief pathway 136 can include the relief contacts (e.g., at least first relief contact 138A and second relief contact 138B) present along one or multiple physical locations along the tissue interface 120 of the electrosurgical tool 102.

In general, the relief pathway 136 needs to be at a higher default impedance than the stimulating pathway 132 at the tissue interface 120 of the electrosurgical tool 102. As voltage builds in the tissue adjacent to the stimulating contacts (e.g., at least first stimulating contact 134A and second stimulating contact 134B) due to coagulum buildup, the impedance through the stimulating pathway increases and prevents effective application of the electrosurgical waveform. The relief pathway 136 permits an escape pathway that is utilized progressively as voltage buildup in the tissue increases and the relief pathway 136 to ground becomes a more electrically advantageous pathway. This permits the current applied through the electrosurgical waveform to spread along the tissue interface 120 of the electrosurgical tool 102 and thereby avoiding potentially dangerous permeation of the current within deeper tissue (e.g., tissue that is distal to the tissue interface 120).

In some examples, the relief pathway 136 could be available continuously under a variable or fixed impedance (e.g., enforced or otherwise exhibited by the impedance component(s) 170). Additionally, relief contacts (e.g., at least first relief contact 138A and second relief contact 138B) of the relief pathway 136 can have individually varied or fixed resistances or impedances relative to the stimulating contacts (e.g., at least first stimulating contact 134A and second stimulating contact 134B). Alternatively, this relief pathway 136 could be selectively adjusted to exhibit a low-impedance or high-impedance configuration based on feedback captured or otherwise interpreted by the system 100, such as based on perceived impedance, temperature, current, or force as discussed above. For example, a current observed through the relief pathway 136 can prompt one or more responses such as further modification of one or more parameters of the electrosurgical waveform. Modification of one or more parameters can include such as voltage enhancement or reduction, or modulating a CF of the electrosurgical waveform. As such, the relief pathway 136 and associated hardware can support or otherwise perform functionalities associated with tissue impedance monitoring.

### Impedance Components

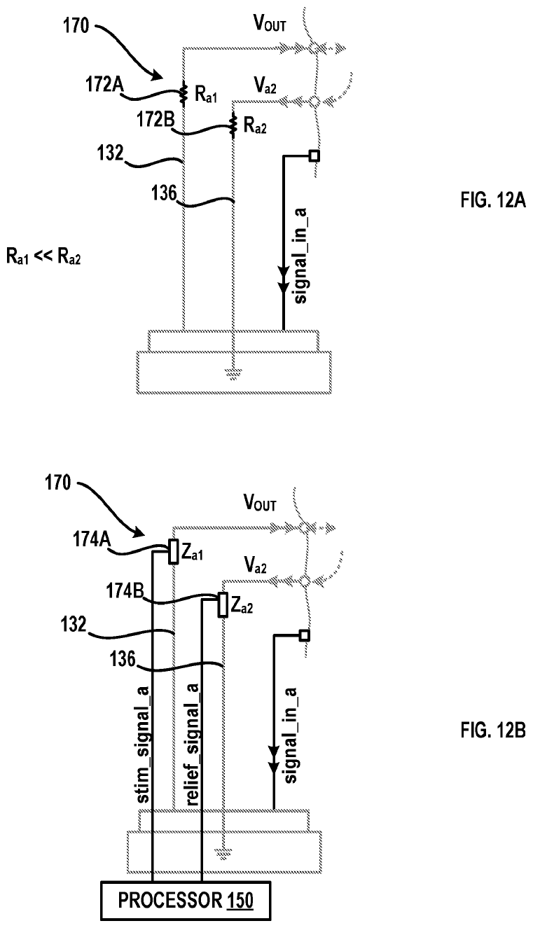

As further shown in FIGS. 10A-11F, and with additional reference to FIGS. 12A and 12B, the electrosurgical tool 102 can include the impedance component(s) 170 that introduce a relief path impedance along the relief pathway 136 to selectively direct current through the stimulating pathway 132 and/or the relief pathway 136. Corresponding with the examples of FIGS. 10A-11F, the first stimulating contact 134A can be associated with a first stimulating pathway impedance Zₐ₁ and the second stimulating contact 134B can be associated with a second stimulating pathway impedance Z_{b1}. The values of the first stimulating pathway impedance Zₐ₁ and the second stimulating pathway impedance Z_{b1} can be variable, fixed, representative of line impedances associated with either side of the stimulating pathway 132 and/or representative of expected impedances associated with tissue 10 captured at the electrosurgical tool 102. Likewise, the first relief contact 138A can be associated with a first relief pathway impedance Zₐ₂ and the second relief contact 138B can be associated with a second relief pathway impedance Z_{b2}. The impedance component(s) 170 can provide the first relief pathway impedance Zₐ₂ and the second relief pathway impedance Z_{b2}. The values of the first relief pathway impedance Zₐ₂ and the second relief pathway impedance Z_{b2} can be variable or fixed as discussed herein. Importantly, the values of the first relief pathway impedance Zₐ₂ and the second relief pathway impedance Z_{b2} can be larger than those of the first stimulating pathway impedance Zₐ₁ and the second stimulating pathway impedance Z_{b1} during unobstructed operation of the electrosurgical tool 102. This directs electrical current through the stimulating pathway 132, especially through tissue captured at the electrosurgical tool 102. As illustrated in the example of FIG. 10F, impedance component(s) 170 can also be used to selectively activate or deactivate associated contacts.

FIG. 12A shows one example implementation of the impedance component(s) 170 including fixed resistors. In this example, a first resistive component 172A denoted as Rai (corresponding with first stimulating pathway impedance Zₐ₁) and associated with the stimulating pathway 132 can be a fixed resistor. The first resistive component 172A denoted as Rₐ₁ can also be representative of line impedances associated with either side of the stimulating pathway 132 and/or representative of expected impedances associated with tissue captured at the electrosurgical tool 102. A second resistive component 172B denoted as Rₐ₂ (corresponding with first relief pathway impedance Zₐ₂) and associated with the relief pathway 136 can be a fixed resistor, where Rₐ₁<<Rₐ₂ such that current is forced through the (lower-impedance) stimulating pathway 132 under normal conditions. If/when an impedance of the stimulating pathway 132 increases, current is directed through the relief pathway 136 and to ground or neutral (such as in the cases of heavy coagulation buildup, no tissue captured at the electrosurgical tool 102, and/or encountering a higher-impedance tissue type).

FIG. 12B shows another example implementation of the impedance component(s) 170 including variable impedance components that receive control signals from another component, such as the processor 150. In this example, a first variable impedance component 174A denoted as Zₐ₁ and associated with the stimulating pathway 132 can be a variable resistor, a transistor-based impedance network, or another suitable device. Alternatively, the first variable impedance component 174A shown in FIG. 12B can be representative of line impedances associated with either side of the stimulating pathway 132 and/or representative of expected impedances associated with tissue captured at the electrosurgical tool 102. A second variable impedance component 174B denoted as Zₐ₂ and associated with the relief pathway 136 can be a variable resistor, transistor-based impedance network, or another suitable device. The second variable impedance component 174B (and in some embodiments, the first variable impedance component 174A) can be controlled by input signals from the processor 150 or another component. In some embodiments, the second variable impedance component 174B of the relief pathway 136 can be controlled based on an impedance exhibited at the stimulating pathway 132 (e.g., as opposed to relying on control signals from a device such as the processor 150)

Similarly, under normal operating conditions corresponding to FIGS. 10A and 11A, Zₐ₁ Zₐ₂ such that current is forced through the (lower-impedance) stimulating pathway 132 under normal conditions. If/when an impedance of the stimulating pathway 132 increases such as in the scenarios shown in FIGS. 10B and 11B, or in FIGS. 10C and 11C, current is directed through the relief pathway 136 and to ground. In some embodiments, the impedance value Zₐ₂ of the second variable impedance component 174B can decrease in response to high impedance at the stimulating pathway 132 to encourage current flow current is directed through the relief pathway 136 and to ground. Further, the impedance value Zₐ₁ of the first variable impedance component 174A can increase to prevent current flow through the stimulating pathway 132. If/when the stimulating pathway impedance returns to an acceptable value, then the impedance value Zₐ₂ of the second variable impedance component 174B can increase and the impedance value Zₐ₁ of the first variable impedance component 174A can decrease to encourage current flow through the stimulating pathway 132.

### Accessory Ground and Relief Pathway

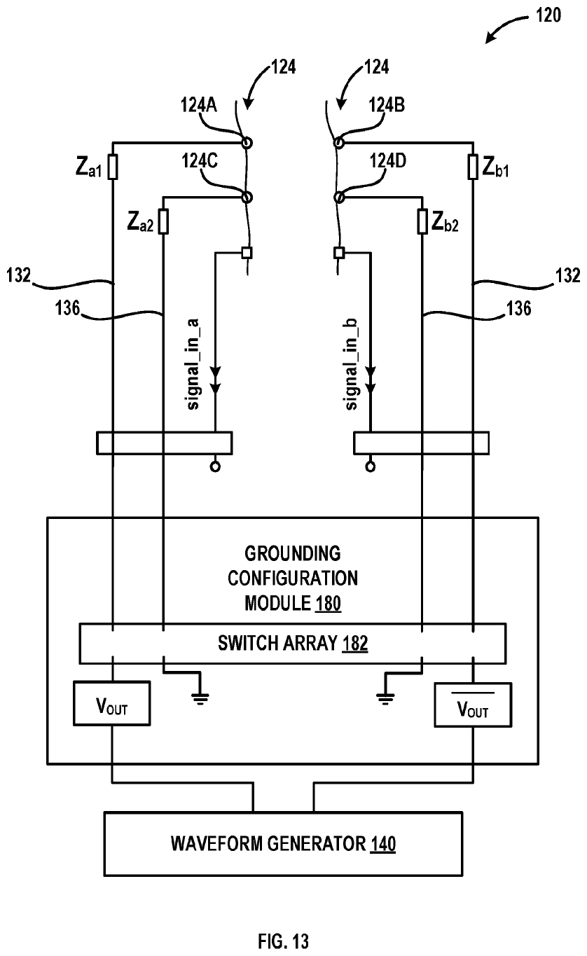

FIG. 13 shows one example configuration of the grounding configuration module 180 that can be used to toggle role assignments for one or more contacts 124 of the electrosurgical tool 102 (e.g., including the stimulating contacts 134A and 134B and the relief contacts 138A and 138B shown in FIGS. 10A-10E). Adjusting the “grounding configuration” and role assignments by the grounding configuration module 180 re-configures the relief pathway 136 and the stimulating pathway 132. As shown, the grounding configuration module 180 can communicate with the waveform generator 140 to receive the electrosurgical waveform (including the first electrosurgical waveform indicated by V_{OUT_1} and the second electrosurgical waveform indicated by V_{OUT_1}). The grounding configuration module 180 can also communicate with the tissue interface 120 of the electrosurgical tool 102 to re-configure a grounding configuration of the contacts 124. In some examples, the grounding configuration module 180 can include a switch array 182 that selectively connects the contacts 124 to one or more outputs of the waveform generator 140 or one or more ground or neutral terminals. The grounding configuration module 180 can, in some embodiments, receive control signals from a processor (e.g., processor 150 shown in FIG. 9) or another component, such as a user interface (e.g., user interface 190 which can be positioned along the electrosurgical tool 102 as shown in FIG. 9).

For example, the grounding configuration module 180 can assign a first contact 124A of the contacts 124 to become the first stimulating contact by selectively connecting the first contact 124A with a first output line of the waveform generator 140 associated with the first electrosurgical waveform. Concurrently, the grounding configuration module 180 can assign a second contact 124B of the contacts 124 to become the second stimulating contact by selectively connecting the second contact 124B with a second output line of the waveform generator 140 associated with the second electrosurgical waveform. Further, the grounding configuration module 180 can assign a third contact 124C and a fourth contact 124D of the contacts 124 to respectively become the first relief contact and the second relief contact by selectively connecting the third contact 124C and the fourth contact 124D with a neutral or ground voltage. To re-configure, the grounding configuration module 180 can toggle role assignments accordingly. For example, the grounding configuration module 180 can connect the second contact 124B and the third contact 124C with the first and second output lines of the waveform generator 140. Likewise, the grounding configuration module 180 can connect the first contact 124A and the fourth contact 124D with the neutral or ground voltage to alternate roles of the contacts. The grounding configuration module 180 can also be configured to selectively toggle or otherwise re-assign roles to any quantity of contacts in this manner. In some use cases, periodically alternating roles of the contacts 124 of the electrosurgical tool 102 can help the electrosurgical tool 102 to remain effective in the presence of coagulum buildup.

### Feedback-Responsive Irrigation

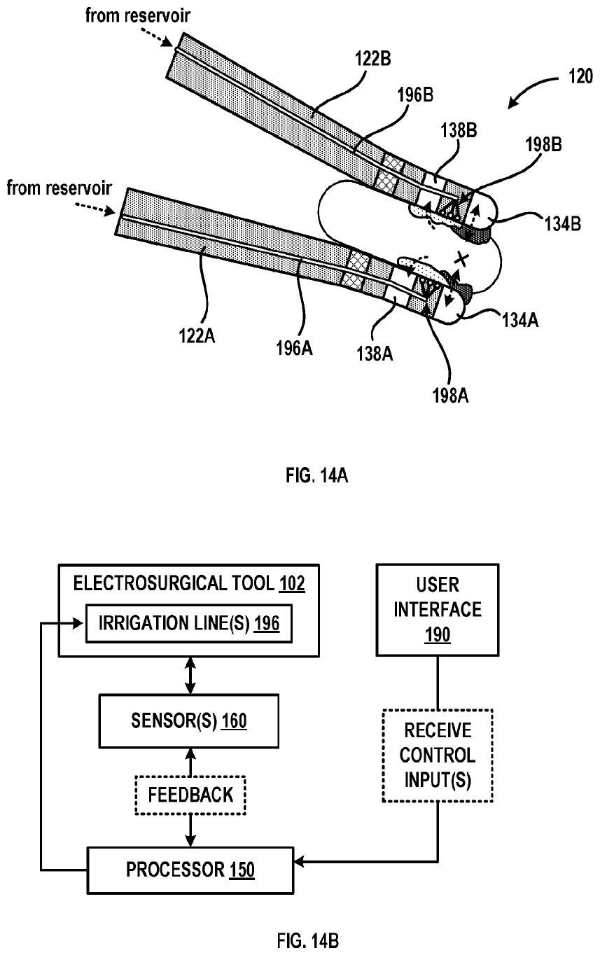

FIGS. 14A and 14B show one embodiment of the tissue interface 120 including irrigation line(s) 196 in fluid flow communication with a first irrigation outlet 198A and a second irrigation outlet 198B for irrigating the surgical space during an electrosurgical task. The irrigation line(s) 196 can introduce water, saline, or another suitable liquid into the surgical space as needed, and can connect to a reservoir (not shown) that stores liquid for irrigation. FIG. 14A shows a first irrigation line 196A in communication with the first irrigation outlet 198A belonging to the first tine 122A, and a second irrigation line 196B in communication with the second irrigation outlet 198B belonging to the second tine 122B. In the example shown, the first irrigation outlet 198A can be positioned between the first stimulating contact 134A and the first relief contact 134B; likewise, the second irrigation outlet 198B can be positioned between the second stimulating contact 134B and the second relief contact 134B. Further, while FIG. 14A shows one particular arrangement with respect to the tissue interface 120, other embodiments are also contemplated in which the first irrigation outlet 198A and the second irrigation outlet 198B are positioned elsewhere along the tissue interface of the electrosurgical tool.

In some examples, with reference to FIG. 14B, the irrigation line(s) 196 can introduce a liquid into the surgical space based on feedback received from sensor(s) 160 at the processor 150. For example, irrigation may be triggered if feedback indicates an exceedingly high temperature measured at tissue, or if there is a significant amount of coagulum buildup at the tissue interface 120 indicated by a high impedance at the stimulating pathway. The irrigation line(s) 196 can also introduce liquid into the surgical space based on control signals received at the user interface 190.

In a further aspect, the irrigation line(s) 196 can introduce a liquid into the surgical space to improve or otherwise facilitate electrical communication between contacts on the same tine. For example, the first irrigation line 196A can introduce liquid into the surgical space at the first irrigation outlet 198A that facilitates electrical communication between the first stimulating contact 134A and the first relief contact 134B.

### Contact Arrangements of Electrosurgical Tool

Further, in some embodiments, the relief contacts and/or the stimulating contacts of the contacts 124 can be selectively adjusted based on the feedback. For example, the relief contacts and/or the stimulating contacts of one or more contacts 124 can have fixed role assignments or can “switch” roles as needed based on the feedback discussed above. The ratio of relief contacts to stimulating contacts can also be variable or fixed.

The contact shape and size can vary within the system 100 such that one or more contacts 124 can occupy any suitable surface area percentage of the tissue interface 120 of the electrosurgical tool 102. The standard shape of a contoured and low profile electrosurgery device (FIGS. 2A and 2B) can be adopted, or alternatively other shapes that permit larger surface areas of tissue-hardware contact could be adopted.

While the examples shown in FIGS. 2A, 2B and 10A-10C show one configuration of the electrosurgical tool 102, other embodiments and arrangements are also contemplated. For example, the electrosurgical tool 102 can have a plurality of stimulating pathways 132 and/or a plurality of relief pathways 136 for focused delivery of the electrosurgical waveform to tissue. Individual contacts of the tissue interface 120 assigned to the stimulating pathway(s) 132 and/or relief pathway(s) 136 can also be arranged in a variety of ways, such as radial arrangements or grid arrangements.

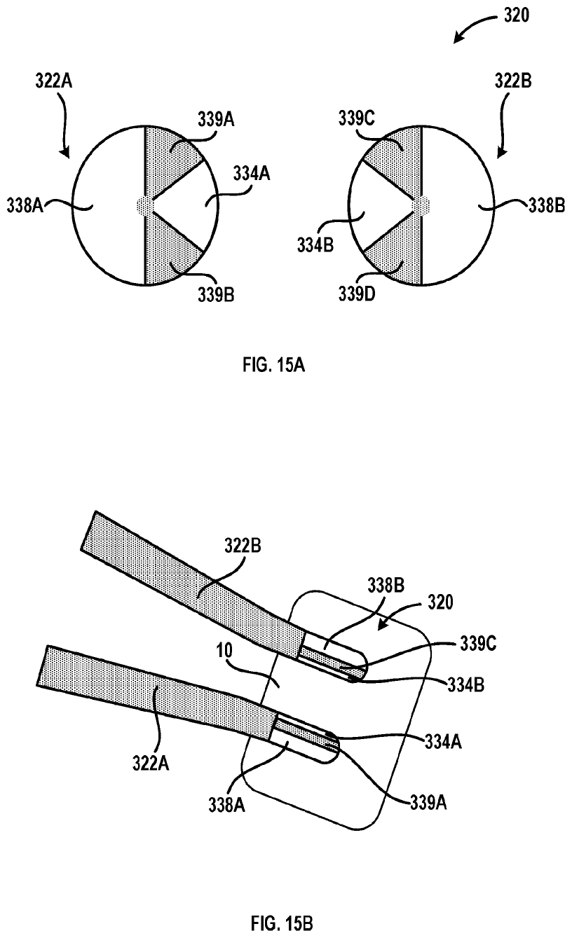

FIGS. 15A and 15B show an alternative, non-limiting arrangement of a tissue interface 320 of an electrosurgical tool showing a first tine 322A and a second tine 322B. In this example, the first tine 322A and the second tine 322B each include at least one stimulating contact and at least one relief contact, where the relief contact(s) are positioned along an external surface of their respective tines. In particular, the first tine 322A includes a first stimulating contact 334A and a first relief contact 338A. The first stimulating contact 334A is positioned along an internal surface of the first tine 322A and the first relief contact 338A is positioned along an external surface of the first tine 322A. In one embodiment, the first relief contact 338A can occupy ˜50% of the surface area of the first tine 322A, along the external surface as shown. The first stimulating contact 334A can occupy ˜20% of the surface area of the first tine 322A, along the internal surface as shown. The remaining ˜30% of surface area of the first tine 322A can be occupied by an insulating material as shown, and can be divided into equal portions (e.g., a first insulating portion 339A occupying ˜15% of the surface area of the first tine 322A along a “front” surface, and a second insulating portion 339B occupying ˜15% of the surface area of the first tine 322A along a “rear” surface).

Likewise, the second tine 322B includes a second stimulating contact 334B and a second relief contact 338B. The second stimulating contact 334B is positioned along an internal surface of the second tine 322B and the second relief contact 338B is positioned along an external surface of the second tine 322B. In one embodiment, the second relief contact 338B can occupy ˜50% of the surface area of the second tine 322B, along the external surface as shown. The second stimulating contact 334B can occupy ˜20% of the surface area of the second tine 322B, along the internal surface as shown. The remaining ˜30% of surface area of the second tine 322B can be occupied by an insulating material as shown, and can be divided into equal portions (e.g., a third insulating portion 339C occupying ˜15% of the surface area of the second tine 322B along a “front” surface, and a second insulating portion 339D occupying 15% of the surface area of the second tine 322B along a “rear” surface).

This arrangement can enable an alternative relief circuit pathway, and can be especially helpful for managing propagation of electrical energy throughout a volume of tissue surrounding the tissue interface 320. In some embodiments, the electrosurgical tool can cycle between which contacts are assigned to stimulating roles or relief roles using time-varying impedance shifting to provide “cool down” intervals. Note that the above surface area allocations (e.g., ˜50% for relief contacts, ˜20% for stimulating contacts, ˜15% for insulating portion) are descriptive of one particular embodiment and that other surface area allocation values are possible. Further, each respective tine may include a plurality of relief contacts, stimulating contacts, and insulating portions. In these examples, the surface area allocations may be divided accordingly (e.g., for a tine having two relief contacts that account for ˜50% of surface area along a tissue interface of the tine, each respective relief contact can occupy ˜25% of surface area along the tissue interface) and may be arranged as appropriate along the tissue interface. For example, contacts of the same role assignment along the same tine may be positioned equidistant from one another with contacts of the opposite role assignment or insulating materials spaced between.

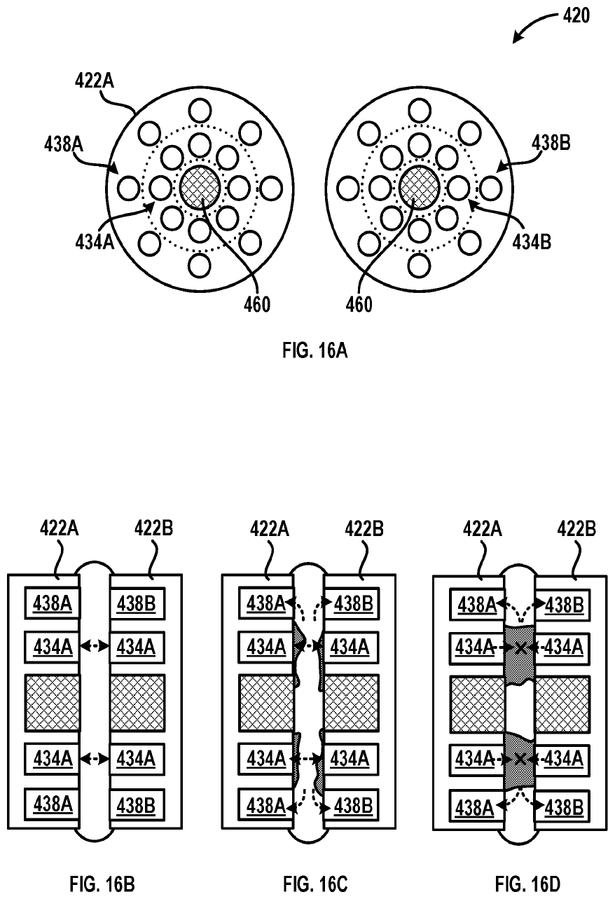

FIGS. 16A-16D show an alternative, non-limiting arrangement of a tissue interface 420 of an electrosurgical tool showing a first tine 422A and a second tine 422B. In this example, the first tine 422A and the second tine 422B each include a plurality of stimulating contacts and a plurality of relief contacts. In FIG. 16A, the first tine 422A includes a first plurality of stimulating contacts 434A and the second tine 422B includes a second plurality of stimulating contacts 434B, where each respective first stimulating contact of the first plurality of stimulating contacts 434A pairs with a respective second stimulating contact of the second plurality of stimulating contacts 434B to define a stimulating pathway of a plurality of stimulating pathways. The first tine 422A can include a first plurality of relief contacts 438A and the second tine 422B can include a second plurality of relief contacts 438B that collectively define a plurality of relief pathways. Sensors 460 can be positioned along the first tine 422A and/or the second tine 422B as shown. Further, each respective contact can be sufficiently separated from other contacts by an insulating material to avoid shorting between respective contacts.

FIG. 16B corresponds to the scenario outlined in FIG. 10A, where there is little to no excessive impedance between the first plurality of stimulating contacts 434A and the second plurality of stimulating contacts 434B of the tissue interface 420 such that the plurality of stimulating pathways are largely unimpeded. FIG. 16C corresponds to FIG. 10B, where there is increased impedance between the first plurality of stimulating contacts 434A and the second plurality of stimulating contacts 434B of the tissue interface 420 due to mild coagulum buildup. As such, some current is diverted through the plurality of relief pathways. FIG. 16D corresponds to FIG. 10C, where there is excessive impedance between the first plurality of stimulating contacts 434A and the second plurality of stimulating contacts 434B of the tissue interface 420 due to excessive coagulum buildup. As such, most or all current is diverted through the plurality of relief pathways.

In some examples, especially for radial multi-contact configurations such as the example shown in FIGS. 16A-16D, subsets of stimulating contacts and relief contacts can be selectively activated or deactivated (e.g., by varying impedances, or through another method such as electrically decoupling). For example, at a first point in time, one stimulating contact of the first plurality of stimulating contacts 434A and one stimulating contact of the second plurality of stimulating contacts 434B may be active and delivering the electrosurgical waveform to tissue as a pair. At the first point in time, remaining stimulating contacts can remain inactive or can be assigned to be relief contacts (e.g., such that there may be two contacts acting as stimulating contacts and more than two contacts acting as relief contacts). Continuing with this example, at a second point in time, another two contacts may be selected as a new pair of stimulating contacts and the originally-assigned stimulating contacts from the first point in time may be deactivated and/or re-assigned to be relief contacts. In another example, two or more pairs may be selected to act as stimulating contacts while the remaining pairs may be selected to act as relief contacts. In this example, the two pairs acting as stimulating contacts may be positioned in an alternating arrangement between the pairs acting as relief contacts (e.g., to ensure even distribution of energy). Re-assignment may be periodic, and may continue in a radial pattern and/or an alternating pattern. In some examples, periodic re-assignment may occur frequently at very short intervals such that an effective surface area of the tissue interface can be maximized or minimized as needed.

Further, while the examples shown in FIGS. 16A-16D show relief contacts being along an outer radial portion of the tissue interface 420 and stimulating contacts being along an inner radial portion of the tissue interface 420. Sensors 460 can be positioned at a center of the tissue interface 420, other arrangements are also contemplated. For example, the contacts along the tissue interface 420 may be arranged in a different manner, such as positioning sensors along an outer radial portion, positioning relief contacts along an inner radial portion, and positioning stimulating contacts at a center of the tissue interface 420. In another example, the contacts along the tissue interface 420 may have fixed or variable roles, and may be positioned along any portion of the tissue interface 420. Further, the contacts along the tissue interface 420 can be operable for assuming any stimulating, relief, or sensing role (e.g., by adjusting a grounding configuration as discussed above with respect to FIG. 9 or by another method).

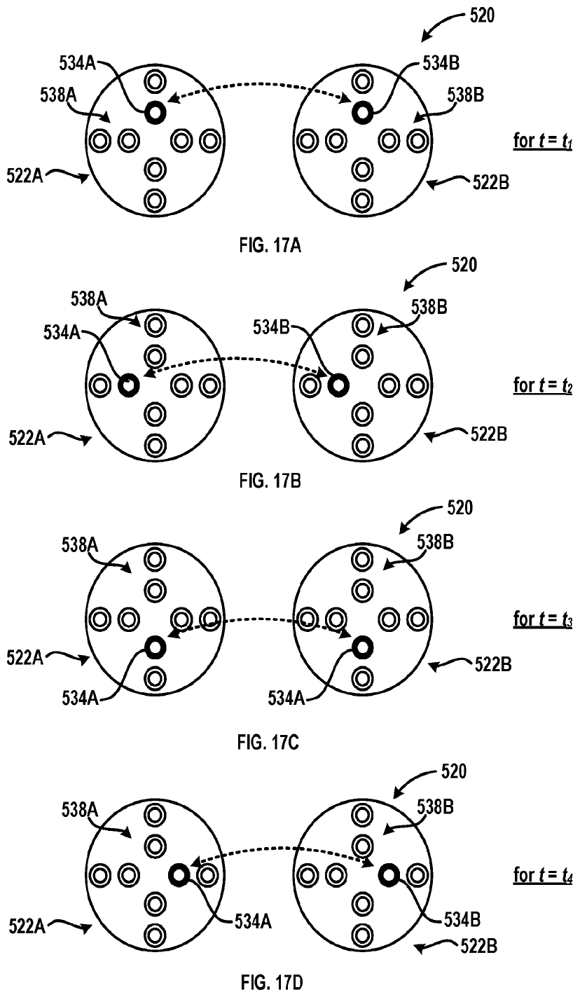

FIGS. 17A-17D show time-varying reconfiguration of contact role assignments for four instances in time (e.g., where FIG. 17A corresponds to t=t₁, FIG. 17B corresponds to t=t₂, FIG. 17C corresponds to t=t₃, and FIG. 17D corresponds to t=t₄). As shown, a tissue interface 520 includes a plurality of contacts across a first tine 522A and a second tine 522B, where each respective tine includes at least one contact assigned as a stimulating contact. In particular, the first tine 522A can include a first stimulating contact 534A of a stimulating pathway and a first plurality of relief contacts 538B of one or more relief pathways. Likewise, the second tine 522B can include a second stimulating contact 534B of the stimulating pathway and a second plurality of relief contacts 538B of one or more relief pathways. In the examples, the first stimulating contact 534A can associate with the second stimulating contact 534B for delivery of the electrosurgical waveform, and the first and second pluralities of relief contacts 538A and 538B can be configured to divert electrical charge away from tissue. Upon re-assignment, a contact that was previously designated as the first stimulating contact 534A or the second stimulating contact 534B can become a relief contact. Re-assignment may be achieved using the ground reconfiguration strategies outlined herein with respect to FIG. 9.

The progression shown in FIGS. 17A-17D show one non-limiting example of how the contact role assignments can alternate over time as needed. In some examples, more than one contact belonging to each respective tine may be assigned to a stimulating contact role. Benefits of alternating contact role assignments can include providing “cool down” time for tissues and contacts, reducing coagulum buildup, diverting electrical charge away from volumetric areas of tissue, and modulating an effective surface area of the tissue interface.

### Methods

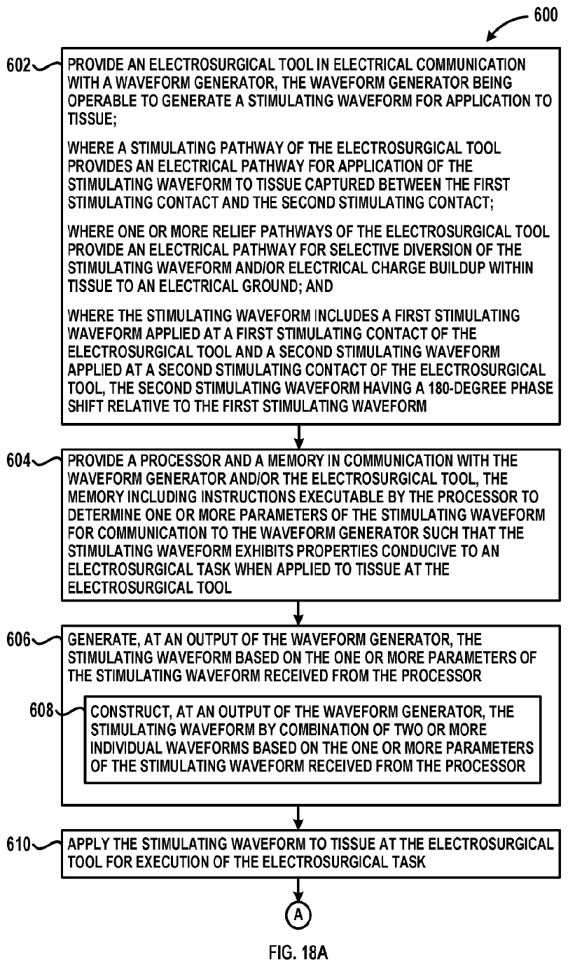

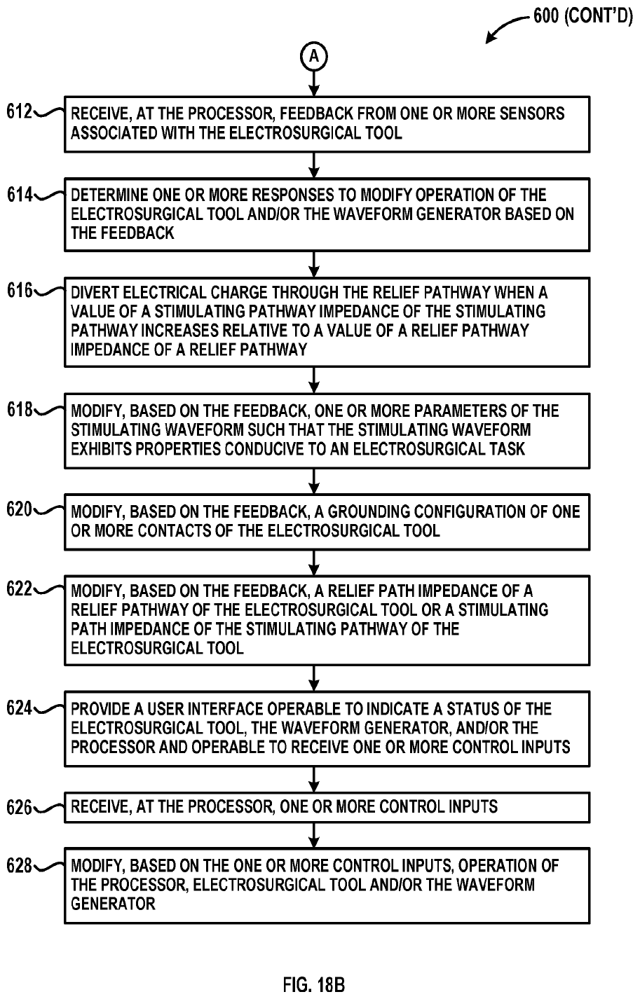

FIGS. 18A and 18B show a method 600 for generating and applying an electrosurgical waveform for an electrosurgical task by the system outlined herein. Note that while the various steps of method 600 are shown in one particular order, it is appreciated that the various steps of method 600 can be applied in any suitable order and may be duplicated or omitted as needed.

As shown in FIG. 18A, step 602 of method 600 includes providing an electrosurgical tool in electrical communication with a waveform generator, the waveform generator being operable to generate an electrosurgical waveform for application to tissue. A stimulating pathway of the electrosurgical tool provides an electrical pathway for application of the electrosurgical waveform to tissue captured between the first stimulating contact and the second stimulating contact. One or more relief pathways of the electrosurgical tool provides an electrical pathway for selective diversion of the electrosurgical waveform and/or electrical charge buildup within tissue to an electrical ground The electrosurgical waveform includes a first electrosurgical waveform applied at a first stimulating contact of the electrosurgical tool and a second electrosurgical waveform applied at a second stimulating contact of the electrosurgical tool. In some examples, the second electrosurgical waveform has a 180-degree phase shift relative to the first electrosurgical waveform.

Step 604 of method 600 includes providing a processor and a memory in communication with the waveform generator and/or the electrosurgical tool, the memory including instructions executable by the processor to determine one or more parameters of the electrosurgical waveform for communication to the waveform generator such that the electrosurgical waveform exhibits properties conducive to an electrosurgical task when applied to tissue at the electrosurgical tool. Step 604 can encompass the step taken by the processor provided in step 604, including determining one or more parameters of the electrosurgical waveform for communication to the waveform generator such that the electrosurgical waveform exhibits properties conducive to an electrosurgical task when applied to tissue at the electrosurgical tool. One or more parameters of the electrosurgical waveform can affect one or more electrosurgical properties of the electrosurgical waveform such that optimal selection of the parameters results in the electrosurgical waveform being conducive for an electrosurgical task. These parameters can include, but are not limited to, a crest factor, a peak-to-peak magnitude, an amplitude, a frequency, and/or a shape of the electrosurgical waveform. In some examples, the electrosurgical waveform can be constructed from a combination of two or more individual waveforms, where each individual waveform can be uniquely associated with parameters including, but not limited to, a crest factor, a peak-to-peak magnitude, an amplitude, a frequency, and/or a shape of the electrosurgical waveform.

Step 606 of method 600 shown in FIG. 18A includes generating, at an output of the waveform generator, the electrosurgical waveform based on one or more parameters of the electrosurgical waveform received from the processor. Step 606 can encompass step 608 of method 600, which includes constructing, at an output of the waveform generator, the electrosurgical waveform by combination of two or more individual waveforms based on the parameters of the electrosurgical waveform received from the processor. Step 610 of method 600 can include applying the electrosurgical waveform to tissue at the electrosurgical tool for execution of the electrosurgical task. FIG. 18A ends at circle A of FIG. 18A.

FIG. 18B continues at circle A of FIG. 18B. As shown, step 612 of method 600 can include receiving, at the processor, feedback from one or more sensors associated with the electrosurgical tool. Step 614 of method 600 can include determining one or more responses to modify operation of the electrosurgical tool and/or the waveform generator based on the feedback. Steps 616-622 of method 600 outline various responses that may be applied by the system for modifying operation based on feedback. Step 616 includes diverting electrical charge through a relief pathway when a value of a stimulating pathway impedance of the stimulating pathway increases relative to a value of a relief pathway impedance of the relief pathway. Step 618 includes modifying, based on the feedback, one or more parameters of the electrosurgical waveform such that the electrosurgical waveform exhibits properties conducive to an electrosurgical task. Step 620 includes modifying, based on the feedback, a grounding configuration of one or more contacts of the electrosurgical tool. Step 622 includes modifying, based on the feedback, a relief path impedance of a relief pathway of the electrosurgical tool or a stimulating path impedance of the stimulating pathway of the electrosurgical tool.

Step 624 of method 600 shown in FIG. 18B includes providing a user interface operable to indicate a status of the electrosurgical tool, the waveform generator, and/or the processor and operable to receive one or more control inputs. Step 626 of method 600 includes receiving, at the processor, one or more control inputs. Step 628 of method 600 includes modifying, based on the control inputs, operation of the processor, electrosurgical tool and/or the waveform generator. Step 628 can encompass steps that are similar to those outlined in steps 616-622, however the modification can be made based on the control inputs (e.g., either in addition to feedback or in lieu of feedback).

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

An example use case for clinical application of this system can be an intracranial arteriovenous malformation resection. The surgeon knows the region of interest that needs to be resected from the brain, however, there are vessels within the region that will need to be preserved and careful coagulation will be necessary to minimize the injury to normal brain and carefully preserve the vessels that pass through the region of the arteriovenous malformation that provide blood to the normal brain. An electrosurgery device that provide precise control of electric waveform penetration within the treatment zone is desired. As the electrosurgery device is used on cerebral parenchyma or on cerebral arteries, arterioles, capillaries, venules, or veins the device can optimize the waveform for application to that tissue type as sensed by impendence. This device can also be used for other delicate surgical needs such as: skull base tumors, cerebral aneurysms, or intraparenchymal tumors. The goals of electrosurgical device parameter selection would be different when comparing an intraparenchymal tumor case where broad surface area hemostasis is the goal, as compared to cerebral aneurysm surgery where precise electrocautery needs to be applied to adhesions or small arteriotomies that are generated during dissection.

### Computer-Implemented Device

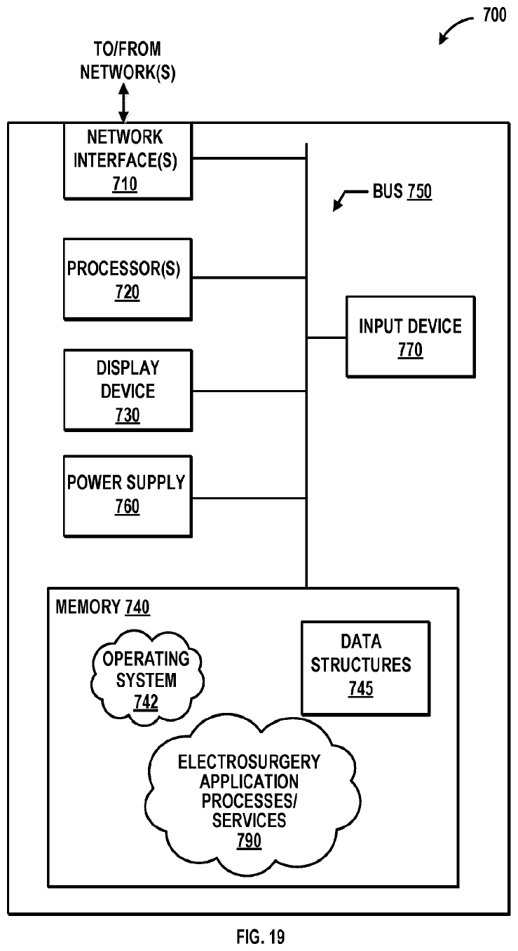

FIG. 19 is a schematic block diagram of an example device 700 that may be used with one or more embodiments described herein, e.g., as a component of the system 100 and any alternate embodiments shown in FIGS. 1-16D and applying aspects of method 600 shown in FIGS. 17A and 17B.

Device 700 comprises one or more network interfaces 710 (e.g., wired, wireless, PLC, etc.), at least one processor 720, and a memory 740 interconnected by a system bus 750, as well as a power supply 760 (e.g., battery, plug-in, etc.). Device 700 can also include a display device 730 in communication with the processor for displaying information (e.g., status information, etc.) about operation of one or more components of the system 100, and an input device 770 for receipt of one or more control inputs (e.g., parameters, commands, etc.) for control of one or more components of the system 100.

Network interface(s) 710 include the mechanical, electrical, and signaling circuitry for communicating data over the communication links coupled to a communication network. Network interfaces 710 are configured to transmit and/or receive data using a variety of different communication protocols. As illustrated, the box representing network interfaces 710 is shown for simplicity, and it is appreciated that such interfaces may represent different types of network connections such as wireless and wired (physical) connections. Network interfaces 710 are shown separately from power supply 760, however it is appreciated that the interfaces that support PLC protocols may communicate through power supply 760 and/or may be an integral component coupled to power supply 760.

Memory 740 includes a plurality of storage locations that are addressable by processor 720 and network interfaces 710 for storing software programs and data structures associated with the embodiments described herein. In some embodiments, device 700 may have limited memory or no memory (e.g., no memory for storage other than for programs/processes operating on the device and associated caches). Memory 740 can include instructions executable by the processor 720 that, when executed by the processor 720, cause the processor 720 to implement aspects of the system 100 and the method 600 outlined herein.

Processor 720 comprises hardware elements or logic adapted to execute the software programs (e.g., instructions) and manipulate data structures 745. An operating system 742, portions of which are typically resident in memory 740 and executed by the processor, functionally organizes device 700 by, inter alia, invoking operations in support of software processes and/or services executing on the device. These software processes and/or services may include electrosurgery application processes/services 790, which can include aspects of method 600 and/or implementations of various modules described herein, and may be implemented in an application that may be operable using a variety of devices. Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a floppy disk, a flexible disk, a hard disk, magnetic tape, any other magnetic medium, a CD-ROM disk, digital video disk (DVD), any other optical medium, RAM, PROM, EPROM, a FLASHEPROM, and any other memory chip or cartridge. Note that while electrosurgery application processes/services 790 is illustrated in centralized memory 740, alternative embodiments provide for the process to be operated within the network interfaces 710, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the electrosurgery application processes/services 790 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

1. A system, comprising:
an electrosurgical tool in electrical communication with a waveform generator;
where the waveform generator is operable to generate an electrosurgical waveform for application to tissue; and
where the electrosurgical tool includes:
a stimulating pathway having a first stimulating contact, where the stimulating pathway provides an electrical pathway for application of the electrosurgical waveform to tissue at the first stimulating contact; and
a relief pathway having a first relief contact, where the relief pathway provides an electrical pathway for selective diversion of the electrosurgical waveform and/or electrical charge buildup within tissue to an electrical ground.

2. The system of claim 1, the electrosurgical tool further comprising:
a second stimulating contact of the stimulating pathway, where the stimulating pathway provides an electrical pathway for application of the electrosurgical waveform to tissue captured between the first stimulating contact and the second stimulating contact;
where the electrosurgical waveform includes:
a first electrosurgical waveform applied at the first stimulating contact of the electrosurgical tool; and
a second electrosurgical waveform applied at the second stimulating contact of the electrosurgical tool.

3. The system of claim 2, the second electrosurgical waveform having a 180-degree phase shift relative to the first electrosurgical waveform.

4. The system of claim 1, where the relief pathway comprises:
an impedance component that introduces a relief pathway impedance along the relief pathway;
wherein a value of the relief pathway impedance is higher than a default value of a stimulating pathway impedance of the stimulating pathway during application of the electrosurgical waveform to tissue; and
wherein an electrical charge is diverted through the relief pathway when a value of the stimulating pathway impedance of the stimulating pathway increases relative to the value of the relief pathway impedance.

5. The system of claim 1, further comprising:
a processor in communication with a memory and the waveform generator, the memory including instructions executable by the processor to:
determine one or more parameters of the electrosurgical waveform such that the electrosurgical waveform exhibits properties conducive to an electrosurgical task; and
communicate the one or more parameters of the electrosurgical waveform to the waveform generator for application to tissue at the electrosurgical tool.

6. The system of claim 5, the memory including instructions executable by the processor to:
determine one or more parameters of two or more individual waveforms that, when combined at an output of the waveform generator to form the electrosurgical waveform, result in the electrosurgical waveform that exhibits properties conducive to the electrosurgical task.

7. The system of claim 1, further comprising:
a processor in communication with a memory, the waveform generator, and the electrosurgical tool, the memory including instructions executable by the processor to:
receive feedback from one or more sensors associated with the electrosurgical tool; and
determine one or more responses to modify operation of the electrosurgical tool and/or the waveform generator based on the feedback.

8. The system of claim 7, the memory including instructions executable by the processor to:
modify, based on the feedback, one or more parameters of the electrosurgical waveform such that the electrosurgical waveform exhibits properties conducive to an electrosurgical task.

9. The system of claim 7, the memory including instructions executable by the processor to:
modify, based on the feedback, a grounding configuration of one or more contacts of the electrosurgical tool.

10. The system of claim 7, the memory including instructions executable by the processor to:
modify, based on the feedback, a relief path impedance of a relief pathway of the electrosurgical tool or a stimulating path impedance of the stimulating pathway of the electrosurgical tool.

11. The system of claim 7, the memory including instructions executable by the processor to:
receive, at the processor, one or more control inputs; and
modify, based on the one or more control inputs, operation of the processor, electrosurgical tool and/or the waveform generator.

12. The system of claim 1, further comprising:
a user interface, including:
one or more display elements operable for indicating a status of the electrosurgical tool, the waveform generator, and/or a processor in communication with the electrosurgical tool and/or the waveform generator.

13. The system of claim 12, the user interface further including:
one or more input elements operable for receiving one or more control inputs;
where receipt of the one or more control inputs at the user interface causes the electrosurgical tool, the waveform generator, and/or a processor in communication with the electrosurgical tool and the waveform generator to apply one or more responses to modify operation of the electrosurgical tool and/or the waveform generator based on the one or more control inputs.

14. The system of claim 1, the electrosurgical waveform being associated with one or more parameters that affect one or more electrosurgical properties of the electrosurgical waveform such that optimal selection of the one or more parameters results in the electrosurgical waveform being conducive for an electrosurgical task;
the one or more parameters of the electrosurgical waveform including a crest factor of the electrosurgical waveform.

15. The system of claim 14, the one or more parameters including a peak-to-peak magnitude of the electrosurgical waveform.

16. The system of claim 14, the one or more parameters including a frequency of the electrosurgical waveform.

17. The system of claim 15, the one or more parameters including a shape of the electrosurgical waveform.

18. A system, comprising:
an electrosurgical tool in electrical communication with a waveform generator;
where the waveform generator is operable to generate a stimulating waveform for application to tissue;
where the electrosurgical tool includes a stimulating pathway having a first stimulating contact and a second stimulating contact, where the stimulating pathway provides an electrical pathway for application of the stimulating waveform to tissue captured between the first stimulating contact and the second stimulating contact; and
where the stimulating waveform includes:
a first stimulating waveform applied at the first stimulating contact of the electrosurgical tool; and
a second stimulating waveform applied at the second stimulating contact of the electrosurgical tool, the second stimulating waveform having a 180-degree phase shift relative to the first stimulating waveform.

19. The system of claim 18, the stimulating waveform being associated with one or more parameters that affect one or more electrosurgical properties of the stimulating waveform such that optimal selection of the one or more parameters results in the stimulating waveform being conducive for an electrosurgical task;
the one or more parameters of the stimulating waveform including a crest factor of the stimulating waveform.

20. The system of claim 18, the electrosurgical tool further comprising:
a relief pathway that includes a first relief contact and a second relief contact, where the relief pathway provides an electrical pathway for selective diversion of the stimulating waveform and/or electrical charge buildup within tissue to an electrical ground.

21. The system of claim 18, further comprising:
a processor in communication with a memory and the waveform generator, the memory including instructions executable by the processor to:
determine one or more parameters of the stimulating waveform such that the stimulating waveform exhibits properties conducive to an electrosurgical task; and
communicate the one or more parameters of the stimulating waveform to the waveform generator for application to tissue at the electrosurgical tool.

22. The system of claim 21, the memory including instructions executable by the processor to:
determine one or more parameters of two or more individual waveforms that, when combined at an output of the waveform generator to form the stimulating waveform, result in the stimulating waveform that exhibits properties conducive to the electrosurgical task.

23. The system of claim 18, further comprising:
a processor in communication with a memory, the waveform generator, and the electrosurgical tool, the memory including instructions executable by the processor to:
receive feedback from one or more sensors associated with the electrosurgical tool; and
determine one or more responses to modify operation of the electrosurgical tool and/or the waveform generator based on the feedback.

24. The system of claim 23, the memory including instructions executable by the processor to:
modify, based on the feedback, one or more parameters of the stimulating waveform such that the stimulating waveform exhibits properties conducive to an electrosurgical task.

25. The system of claim 23, the memory including instructions executable by the processor to:
modify, based on the feedback, a grounding configuration of one or more contacts of the electrosurgical tool.

26. The system of claim 23, the memory including instructions executable by the processor to:
modify, based on the feedback, a relief path impedance of a relief pathway of the electrosurgical tool or a stimulating path impedance of the stimulating pathway of the electrosurgical tool.

27. A method, comprising:
providing an electrosurgical tool in electrical communication with a waveform generator;
where the waveform generator is operable to generate an electrosurgical waveform for application to tissue;
where the electrosurgical tool includes a stimulating pathway including a first stimulating contact, where the stimulating pathway provides an electrical pathway for application of the electrosurgical waveform to tissue at the first stimulating contact; and
a relief pathway having a first relief contact, where the relief pathway provides an electrical pathway for selective diversion of the electrosurgical waveform and/or electrical charge buildup within tissue to an electrical ground; and
providing a processor and a memory in communication with the waveform generator and/or the electrosurgical tool, the memory including instructions executable by the processor to:
determine one or more parameters of the electrosurgical waveform for communication to the waveform generator such that the electrosurgical waveform exhibits properties conducive to an electrosurgical task when applied to tissue at the electrosurgical tool.

28. The method of claim 27, further comprising:
diverting electrical charge through the relief pathway when a value of a stimulating pathway impedance of the stimulating pathway increases relative to a value of a relief pathway impedance of the relief pathway.

29. The method of claim 27, further comprising:
providing the electrosurgical tool in electrical communication with the waveform generator, where the electrosurgical tool includes:
a second stimulating contact of the stimulating pathway, where the stimulating pathway provides an electrical pathway for application of the electrosurgical waveform to tissue captured between the first stimulating contact and the second stimulating contact;
where the electrosurgical waveform includes:
a first electrosurgical waveform applied at the first stimulating contact of the electrosurgical tool; and
a second electrosurgical waveform applied at the second stimulating contact of the electrosurgical tool.

30. The method of claim 29, the second electrosurgical waveform having a 180-degree phase shift relative to the first electrosurgical waveform.

31. The method of claim 27, further comprising:
generating, at an output of the waveform generator, the electrosurgical waveform based on the one or more parameters of the electrosurgical waveform received from the processor.

32. The method of claim 31, further comprising:
constructing, at an output of the waveform generator, the electrosurgical waveform by combination of two or more individual waveforms based on the one or more parameters of the electrosurgical waveform received from the processor.

33. The method of claim 27, further comprising:
receiving, at the processor, feedback from one or more sensors associated with the electrosurgical tool; and
determining one or more responses to modify operation of the electrosurgical tool and/or the waveform generator based on the feedback.

34. The method of claim 33, further comprising:
modifying, based on the feedback, one or more parameters of the electrosurgical waveform such that the electrosurgical waveform exhibits properties conducive to an electrosurgical task.

35. The method of claim 33, further comprising:
modifying, based on the feedback, a grounding configuration of one or more contacts of the electrosurgical tool.

36. The method of claim 33, further comprising:
modifying, based on the feedback, a relief path impedance of the relief pathway of the electrosurgical tool or a stimulating path impedance of the stimulating pathway of the electrosurgical tool.

37. The method of claim 33, further comprising:
receiving, at the processor, one or more control inputs; and
modifying, based on the one or more control inputs, operation of the processor, electrosurgical tool and/or the waveform generator.

38. A non-transitory computer-readable storage medium having instructions embodied thereon, the instructions executable by a computing system to perform a method for generating and applying an electrosurgical waveform for an electrosurgical task, the method comprising:
determining one or more parameters of an electrosurgical waveform for application at an electrosurgical tool such that the electrosurgical waveform exhibits properties conducive to an electrosurgical task;
where the electrosurgical waveform includes:
a first electrosurgical waveform for application at a first stimulating contact of the electrosurgical tool; and
a second electrosurgical waveform for application at a second stimulating contact of the electrosurgical tool, the second electrosurgical waveform having a 180-degree phase shift relative to the first electrosurgical waveform; and
communicating the one or more parameters of the electrosurgical waveform to a waveform generator in communication with the electrosurgical tool for application to tissue at the electrosurgical tool.

39. A method of treatment, comprising:
providing an electrosurgical tool in electrical communication with a waveform generator;
where the waveform generator is operable to generate an electrosurgical waveform for application to tissue;
where the electrosurgical tool includes a stimulating pathway including a first stimulating contact and a second stimulating contact, where the stimulating pathway provides an electrical pathway for application of the electrosurgical waveform to tissue captured between the first stimulating contact and the second stimulating contact; and
where the electrosurgical waveform includes:
a first electrosurgical waveform applied at the first stimulating contact of the electrosurgical tool; and
a second electrosurgical waveform applied at the second stimulating contact of the electrosurgical tool, the second electrosurgical waveform having a 180-degree phase shift relative to the first electrosurgical waveform;
providing a processor and a memory in communication with the waveform generator and/or the electrosurgical tool, the memory including instructions executable by the processor to:
determine one or more parameters of the electrosurgical waveform such that the electrosurgical waveform exhibits properties conducive to an electrosurgical task; and
applying the electrosurgical waveform to tissue at the electrosurgical tool for execution of the electrosurgical task.
